(19) United States
(12) Patent Application Publication
(10) Pub. No.: US 2021/0340189 A1
(43) Pub. Date: Nov. 4, 2021

(54) SARS-COV-2 N PROTEIN AND METHODS OF USE

(71) Applicant: REGENTS OF THE UNIVERSITY OF MINNESOTA, Minneapolis, MN (US)

(72) Inventors: Yuying Liang, Minneapolis, MN (US); Hinh Ly, Minneapolis, MN (US); Da Di, Minneapolis, MN (US)

(21) Appl. No.: 17/245,599

(22) Filed: Apr. 30, 2021

Related U.S. Application Data
(60) Provisional application No. 63/018,151, filed on Apr. 30, 2020.

(51) Int. Cl. C07K 14/165; G01N 33/68; C07K 16/10; C07K 1/113

(52) U.S. Cl. CPC C07K 14/165; G01N 33/6893; A61K 38/00; C07K 1/1136; C07K 16/10

(57) ABSTRACT

Provided herein is a SARS-CoV-2 coronavirus nucleocapsid (N) protein, where the N protein is substantially free of RNA. Also provided are methods of using the N protein including methods for inducing anti-N protein antibody in a subject, treating an infection, and detecting the presence or absence of anti-N protein antibody.

### CROSS-REFERENCE TO RELATED APPLICATIONS

This application claims the benefit of U.S. Provisional Application Ser. No. 63/018,151, filed Apr. 30, 2020, which is incorporated by reference herein in its entirety.

### SEQUENCE LISTING

This application contains a Sequence Listing electronically submitted via EFS-Web to the United States Patent and Trademark Office as an ASCII text file entitled “0110-000658US01_ST25” having a size of 8 kilobytes and created on Apr. 29, 2021. The information contained in the Sequence Listing is incorporated by reference herein.

### SUMMARY

SARS-CoV-2 nucleoprotein (N) binds single-stranded viral RNA genome to form helical ribonucleoprotein complex that is packaged into virion particles. Described herein is the expression and purification from bacterial cells two recombinant forms of SARS-CoV-2 N, one from the soluble fraction of bacterial cell lysates that is strongly associated with bacterial RNAs, N(+RNA), and the other that is completely devoid of detectable RNAs, N(−RNA). Both forms of N can be used to develop enzyme-linked immunosorbent assays (ELISAs) for specific detection of human and mouse anti-N monoclonal antibodies (mAb) as well as feline SARS-CoV-2 seropositive serum samples, but N(−RNA) shows higher sensitivity level than N(+RNA) to detecting anti-N antibody.

Terms used herein will be understood to take on their ordinary meaning in the relevant art unless specified otherwise. Several terms used herein and their meanings are set forth below.

As used herein, an “isolated” substance is one that has been removed from its natural environment, produced using recombinant techniques, or chemically or enzymatically synthesized. For instance, a protein or a polynucleotide can be isolated. In one embodiment, a substance is purified, i.e., is at least 60% free, preferably at least 75% free, and most preferably at least 90% free from other components with which they are naturally associated.

As used herein, the term “polynucleotide” refers to a polymeric form of nucleotides of any length, either ribonucleotides or deoxynucleotides, and includes both double- and single-stranded RNA and DNA. A polynucleotide can be obtained directly from a natural source, or can be prepared with the aid of recombinant, enzymatic, or chemical techniques. A polynucleotide can be linear or circular in topology. A polynucleotide may be, for example, a portion of a vector, such as an expression or cloning vector, or a fragment. A polynucleotide may include nucleotide sequences having different functions, including, for instance, coding regions, and non-coding regions such as regulatory regions.

A polynucleotide that includes a coding region may include heterologous nucleotides that flank one or both sides of the coding region. As used herein, “heterologous nucleotides” refer to nucleotides that are not normally present flanking a coding region. Examples of heterologous nucleotides include but are not limited to regulatory sequences. Typically, heterologous nucleotides are present in a polynucleotide of the present disclosure through the use of standard genetic and/or recombinant methodologies well known to one skilled in the art. A polynucleotide of the present disclosure may be included in a suitable vector.

As used herein, an “exogenous polynucleotide” refers to a polynucleotide that is not normally or naturally found in a cell, such as a microbe. As used herein, the term “endogenous polynucleotide” refers to a polynucleotide that is normally or naturally found in a cell, such as a microbe. An “endogenous polynucleotide” is also referred to as a “native polynucleotide.”

As used herein, the term “protein” refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, tetramers). Thus, the terms peptide, oligopeptide, enzyme, and polypeptide are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, “identity” refers to sequence similarity between two proteins or two polynucleotides. The sequence similarity between two proteins is determined by aligning the residues of the two proteins (e.g., a candidate amino acid sequence and a reference amino acid sequence, such as SEQ ID NO:1) to optimize the number of identical amino acids along the lengths of their sequences; gaps in either or both sequences are permitted in making the alignment in order to optimize the number of shared amino acids, although the amino acids in each sequence must nonetheless remain in their proper order. The sequence similarity between two polynucleotides is determined by aligning the residues of the two polynucleotides (e.g., a candidate nucleotide sequence and a reference polynucleotide sequence, such as SEQ ID NO:2 or 3) to optimize the number of identical nucleotides along the lengths of their sequences; gaps in either or both sequences are permitted in making the alignment in order to optimize the number of shared nucleotides, although the nucleotides in each sequence must nonetheless remain in their proper order. The sequence similarity is typically at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity. Sequence similarity may be determined, for example, using sequence techniques such as the BESTFIT algorithm in the GCG package (Madison Wis.), or the Blastp or Blastn program of the BLAST 2 search algorithm, and available through the World Wide Web, for instance at the internet site maintained by the National Center for Biotechnology Information, National Institutes of Health. Preferably, sequence similarity between two amino acid sequences is determined using the Blastp program of the BLAST 2 search algorithm, and sequence similarity between nucleotide sequences is determined using the Blastn program of the BLAST 2 search algorithm. Preferably, the default values for all BLAST2 search parameters are used.

As used herein, “genetically modified cell” refers to a cell, such as a microbe, into which has been introduced an exogenous polynucleotide, e.g., an expression vector. For example, a microbe is a genetically modified microbe by virtue of introduction into a suitable microbe of an exogenous polynucleotide that is foreign to the microbe.

As used herein, an antibody that can “specifically bind” or is “specific for” a protein is an antibody that interacts only with an epitope of the antigen that induced the synthesis of the antibody, or interacts with a structurally related epitope.

As used herein, “heterologous amino acids” refers to amino acids that are not normally or naturally found flanking a natural SARS-CoV-2 N protein, for instance, the sequence depicted at SEQ ID NO: 1.

As used in this specification and the appended claims, the term “or” is generally employed as including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have”, “has”, “having”, “include”, “includes”, “including”, “comprise”, “comprises”, “comprising” and the like are used in their open ended inclusive sense, and generally mean “include, but not limited to”, “includes, but not limited to”, or “including, but not limited to”.

It is understood that wherever embodiments are described herein with the language have”, “has”, “having”, “include”, “includes”, “including”, “comprise”, “comprises”, “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Unless otherwise specified, “a,” “an,” “the,” and “at least one” are used interchangeably and mean one or more than one.

Conditions that are “suitable” for an event to occur, or “suitable” conditions are conditions that do not prevent such events from occurring. Thus, these conditions permit, enhance, facilitate, and/or are conducive to the event.

As used herein, “substantially free of” a material (including, for example, RNA associated with N protein) refers to a protein or a composition having less than 10% of the material, less than 5% of the material, less than 4% of the material, less than 3% of the material, less than 2% of the material, or less than 1% of the material. In one embodiment, the presence of the material in a composition is undetectable.

As used herein, “providing” in the context of, for instance, a protein, composition, or a cell means making the protein, composition, or a cell, purchasing the protein, composition, or a cell, or otherwise obtaining the protein, composition, or a cell.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible Subranges as well as individual numerical values within that range. For example, description of a range such as from 1 to 6 should be considered to have specifically disclosed Subranges Such as from 1 to 3, from 1 to 4, from 1 to 5, from 2 to 4, from 2 to 6, from 3 to 6 etc., as well as individual numbers within that range, for example, 1, 2, 2.7.3, 4, 5, 5.3, and 6. This applies regardless of the breadth of the range.

For any method disclosed herein that includes discrete steps, the steps may be conducted in any feasible order. And, as appropriate, any combination of two or more steps may be conducted simultaneously

### BRIEF DESCRIPTION OF THE FIGURES

The following detailed description of illustrative embodiments of the present disclosure may be best understood when read in conjunction with the following drawings.

FIGS. 1A-B show expression and purification of recombinant SARS-CoV-2 N protein with or without associated bacterial RNAs. Bacterial cell lysates, fractions, wash and elution collections were loaded onto and separated by 12% SDS-PAGE followed by Coomassie blue staining. (FIG. 1A) Expression and purification of the SARS-CoV-2 N protein from the soluble bacterial cell lysates. M, protein markers. U, uninduced cell lysates. Ind, IPTG-induced cell lysates. Ins, insoluble fraction. W, solution after washing the HisTrap column. E1 to E4, elution fractions. (FIG. 1B) Purification of the SARS-CoV-2 N protein using the denature and renature methods. M, protein markers. S, soluble fraction after protein denaturation by urea treatment. Ins, insoluble protein fraction after urea denaturation. F, flow through fraction after applying proteins through the HisTrap column. W1, W3, W5 fraction collections after washing the HisTrap column. E1, E3, E5 fraction collections after elution of the samples from the HisTrap column.

FIG. 2 shows evaluation of the recombinant N(−RNA) and N(+RNA) proteins to detect human anti-SARS-CoV-2 N mAb by a commercial human COVID-19 N IgG/IgM ELISA kit. ELISA plate was coated with N(−RNA) or N(+RNA) protein at indicated concentration(s) per well and incubated with the positive (p) or negative (n) controls supplied by the commercial COVID-19 N IgG/IgM ELISA kit. Results shown are the average of triplicate sets of data at different SARS-CoV-2 N protein concentrations. At least two independent experiments were conducted for each set of experiments. Results obtained from the commercial kit are shown in black bars, those from N(+RNA)-coated wells are shown in blue bars, and those from N(−RNA)-coated wells in red bars. p, positive control; n, negative control.

FIGS. 3A-B show evaluation of N(+RNA) and N(−RNA) proteins in detecting anti-N mAb by ELISA. Serial dilutions of mouse anti-SARS-CoV N mAb were incubated in an ELISA plate pre-coated with 100 ug of either N(−RNA) or N(+RNA) and analyzed by standard ELISA procedure as described in Materials and Methods. (FIG. 3A) The average OD₄₅₀ value of triplicates is plotted against the mAb dilutions for N(−RNA) or N(+RNA). The cut-off value is shown as a dashed line. (FIG. 3B) The N-specific IgG endpoint titers, based on N(−RNA) and N(+RNA), respectively, were calculated as the highest dilution to give an OD₄₅₀ value exceeding the cut-off OD₄₅₀ value.

FIG. 4 shows evaluation of N(+RNA) and N(−RNA) proteins in the N-based ELISA using feline clinical serum samples. Seventeen seropositive (pos) and fourteen seronegative (neg) cat serum samples were analyzed by N-based ELISA with either N(+RNA) and N(−RNA). The cut-off OD₄₅₀ value is shown as a dashed line.

FIGS. 5A-C show recombinant SARS-CoV-2 N protein preferentially binds to ssRNA in vitro. The ssRNA (FIG. 5A) and dsRNA (FIG. 5B) templates were incubated with increasing concentrations of the recombinant N(−RNA) protein and separated in 1% agarose gel with ethidium bromide. (FIG. 5C) The ssRNA template was incubated with either NTD or CTD of the SARS-CoV-2 N protein at increasing concentrations and separated in 1% agarose gel with ethidium bromide. At least two independent experiments were conducted per assay.

FIGS. 6A-E show serological tests of pet cat sera by ELISA. (FIG. 6A) Purified recombinant SARS-CoV-2 N and RBD proteins shown in SDS-PAGE gel after Coomassie blue staining. (FIG. 6B) A representative SARS-CoV-2 N IgG ELISA with pet cat sera. Normal cat serum purchased from a commercial source, the positive control (SARS-CoV N-specific mAb 1C7C7), and two seropositive samples (#29 and #11) are shown. (FIG. 6C) Pet cat sera tested by RBD IgG ELISA. The positive control (mAb 1C7C7), and seropositive samples are shown. (FIG. 6D) A batch of pet cat sera were tested with both N and RBD IgG ELISA. None of the RBD-positive sera are N-negative. The ID # of N seropositive samples are shown. (FIG. 6E) Evaluation of pet cat sera with IgG ELISA against feline infectious peritonitis virus (FIPV) antigens. Each serum was tested pairwise in uncoated and coated wells in technical duplicates. The adjusted OD450 value was calculated by subtracting OD450 value of uncoated well from that of the coated well. The cut-off OD450 value was calculated as described in Example 2 and shown as a dashed line.

FIGS. 7A-B show quantification of SARS-CoV-2 neutralizing antibodies (nAb) in pet cat sera. The SARS-CoV neutralizing assay was conducted using a SARS-CoV-2 S pseudotyped replication-defective VSV expressing the firefly luciferase (FLUC) reporter gene. The FLUC activity was measured at 24 h post-infection and normalized to control wells (and set as 1). Each sample was measured in technical duplicates. (FIG. 7A) The pseudotyped virus entry is dependent on SARS-CoV-2 host receptor hACE2. The neutralization assay was conducted with increasing concentrations of recombinant hACE2. (FIG. 7B) A representative neutralization assay of pet cat serum samples. Two independent experiments were conducted.

FIGS. 8A-C show serological tests of pet dog sera. (FIG. 8A) Validation of the pooled N-based ELISA test. A pool of 5 pet dog samples were tested in the standard dog N-based ELISA as described in Example 2. The negative control (Neg) consists of all 5 N seronegative samples confirmed by previous individual ELISA test. The positive control (1 pos) consists of 4 N seronegative samples and 1 seropositive sample. The cut-off OD450 value is shown in a dashed line. (FIG. 8B) Testing of pet dog sera by the pooled N-based ELISA. The positive control (pos ctrl) consists of one confirmed N seropositive serum and four confirmed N seronegative sera, and a seropositive pool (#45) are shown. (FIG. 8C) Identification of the seropositive pet dog sample in pool #45 by individual N-based ELISA. A positive control (pos ctrl) and the seropositive sample #432 are shown. Each sample was measured in technical duplicates.

FIG. 9 shows two nucleotide sequences encoding an N protein. SEQ ID NO:2 is a coding region optimized for expression in human cells, and SEQ ID NO:3 is a coding region optimized for expression in E. coli.

### DETAILED DESCRIPTION

The present disclosure includes isolated nucleocapsid (N) protein encoded by SARS-CoV-2 virus (also referred to as Covid-19 virus). N protein is typically 418 amino acids in length and is responsible for capturing the genome of coronavirus (CoV) into the virion particle. It has two main structural domains and 3 disordered regions. The first structural domain is located near the N-terminal region from amino acid 40 to 180 and has the ability to bind to single-stranded RNA (ssRNA), hence it is known as the RNA binding domain (Surjit et al., Infect. Genet. Evol. 2008 July; 8(4):397-405. doi: 10.1016/j.meegid.2007.07.004.). The second structural domain is located from amino acid 247 to 364 and is reported to be responsible for forming dimeric structure of CoV N proteins (Surjit et al., Infect. Genet. Evol. 2008 July; 8(4):397-405. doi: 10.1016/j.meegid.2007.07.004.). The three disordered regions of the protein are located with the first region at around amino acids 1-40; the second disordered region located at around amino acids 180-247 between the two structural domains; and the third disordered region located around amino acids 365-418. The third region has been reported to interact with the C-terminal domain of the CoV membrane (M) protein on the virion membrane (Kuo et al., J. Virol. 2016, doi:10.1128/jvi.03212-15).

N protein has biological activity. In one embodiment, N protein has the activity of binding RNA. The RNA binding activity of N protein is a non-specific binding, thus the sequence of the RNA is irrelevant. In one embodiment, N protein has immunological activity. “Immunological activity” refers to the ability of N protein to elicit an immunological response in a subject. An immunological response to a protein is the development in a subject of a cellular and/or antibody-mediated immune response to the protein. Usually, an immunological response includes but is not limited to one or more of the following effects: the production of antibodies, B cells, helper T cells, suppressor T cells, and/or cytotoxic T cells, directed to an epitope or epitopes of the protein. “Epitope” refers to the site on a N protein to which specific B cells and/or T cells respond so that antibody is produced. The immunological activity may be protective. “Protective immunological activity” refers to the ability of a N protein to elicit an immunological response in a subject that inhibits or limits infection by SARS-CoV-2 virus. Whether N protein has protective immunological activity can be determined by methods and animal models known in the art. N protein may have seroactive activity. As used herein, “seroactive activity” refers to the ability of N protein to react with antibody present in serum from a subject infected with SARS-CoV-2 virus or antibody present in convalescent serum from a subject infected with SARS-CoV-2 virus.

The amino acid sequence of N protein is readily available to the skilled person. An example of a N protein is available at Genbank Accession number YP_009724397 (also referred to herein as SEQ ID NO:1). Other examples of N proteins of the present disclosure include those having sequence similarity with the amino acid sequence of SEQ ID NO:1. A N protein having sequence similarity with the amino acid sequence of SEQ ID NO:1 has biological activity, e.g., RNA binding activity, immunological activity, protective immunological activity, and/or seroactive activity. A N protein may be isolated from a SARS-CoV-2 virus particle, or may be produced using recombinant techniques, or chemically or enzymatically synthesized using routine methods.

The amino acid sequence of a N protein having sequence similarity to SEQ ID NO:1 may include conservative substitutions of amino acids present in SEQ ID NO:1. A conservative substitution is typically the substitution of one amino acid for another that is a member of the same class. For example, it is well known in the art of protein biochemistry that an amino acid belonging to a grouping of amino acids having a particular size or characteristic (such as charge, hydrophobicity, and/or hydrophilicity) may generally be substituted for another amino acid without substantially altering the secondary and/or tertiary structure of a protein. For the purposes of this disclosure, conservative amino acid substitutions are defined to result from exchange of amino acids residues from within one of the following classes of residues: Class I: Gly, Ala, Val, Leu, and Ile (representing aliphatic side chains); Class II: Gly, Ala, Val, Leu, Ile, Ser, and Thr (representing aliphatic and aliphatic hydroxyl side chains); Class III: Tyr, Ser, and Thr (representing hydroxyl side chains); Class IV: Cys and Met (representing sulfur-containing side chains); Class V: Glu, Asp, Asn and Gln (carboxyl or amide group containing side chains); Class VI: His, Arg and Lys (representing basic side chains); Class VII: Gly, Ala, Pro, Trp, Tyr, Ile, Val, Leu, Phe and Met (representing hydrophobic side chains); Class VIII: Phe, Trp, and Tyr (representing aromatic side chains); and Class IX: Asn and Gln (representing amide side chains). The classes are not limited to naturally occurring amino acids, but also include artificial amino acids, such as beta or gamma amino acids and those containing non-natural side chains, and/or other similar monomers such as hydroxyacids.

A protein of the present disclosure may be expressed as a fusion protein that includes a protein described herein and heterologous amino acids. Addition of heterologous amino acids can be achieved by the in-frame addition of a nucleotide sequence encoding the desired domain directly to either the 5′ or 3′ end of a coding region that encodes a protein of the present disclosure. In one embodiment, heterologous amino acids may be a carrier protein. The carrier protein may be used to increase the immunogenicity of the fusion protein to increase production of antibodies that specifically bind to a protein of the disclosure. Such constructs may be useful for the production of vaccines (Song et al., 2008, PLoS ONE, 3(5):e2257). The disclosure is not limited by the types of carrier proteins that may be used to create fusion proteins. Examples of carrier proteins include, but are not limited to, keyhole limpet hemacyanin, bovine serum albumin, ovalbumin, mouse serum albumin, rabbit serum albumin, and the like.

In one embodiment, heterologous amino acids may be an affinity purification tag, such as, but not limited to, a polyhistidine-tag (His-tag) and a strep-tag. Incorporation of an affinity purification tag permits the easy isolation of the protein. Optionally, the tag can then be cleaved. Other suitable affinity purification tags (e.g., maltose-binding protein) and methods of purification of proteins with those tags are known in the art.

In one embodiment, a N protein can be covalently bound to a hapten to improve the immunogenicity of the protein. Useful haptens are known in the art. The chemical coupling of a N protein to a hapten can be carried out using known and routine methods.

The present disclosure also includes isolated polynucleotides encoding a N protein. In one embodiment, a polynucleotide may have a nucleotide sequence encoding a protein having the amino acid sequence shown in SEQ ID NO:1. It is understood that a polynucleotide encoding a N protein represented by SEQ ID NO:1 is not limited to a single sequence, but includes the class of polynucleotides encoding such a protein as a result of the degeneracy of the genetic code. For example, a naturally occurring nucleotide sequence encoding N protein is but one member of the class of nucleotide sequences encoding a protein having the amino acid sequence SEQ ID NO:1. The class of nucleotide sequences encoding a selected protein sequence is large but finite, and the nucleotide sequence of each member of the class may be readily determined by one skilled in the art by reference to the standard genetic code, wherein different nucleotide triplets (codons) are known to encode the same amino acid. In some embodiments, a nucleotide sequence can be optimized for expression in a host cell by reference to a host cell's codon usage bias. A non-limiting example of a nucleotide sequence optimized for expression in human cells is shown in SEQ ID NO:2, and a non-limiting example of a nucleotide sequence optimized for expression in E. coli is shown in SEQ ID NO:3. Accordingly, the present disclosure includes a coding region SEQ ID NO:2 and SEQ ID NO:3; a coding region having sequence similarity of at least 80% identity to SEQ ID NO:2 that encodes the N protein of SEQ ID NO:1 and optionally optimized for expression in either human cells; and a coding region having sequence similarity of at least 80% identity to SEQ ID NO:3 that encodes the N protein of SEQ ID NO:1 and optionally optimized for expression in E. coli cells. Other examples of coding regions include one that encodes the N protein of SEQ ID NO:1 or a N protein having sequence similarity of at least 80% identity to SEQ ID NO:1 and optimized for expression in human cells or in E. coli cells.

A polynucleotide encoding a protein of the present disclosure may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the disclosure employs standard ligation techniques known in the art. See, e.g., Sambrook et al, Molecular Cloning: A Laboratory Manual., Cold Spring Harbor Laboratory Press (1989). A vector may provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, and artificial chromosome vectors. Examples of viral vectors include, for instance, adenoviral vectors, adeno-associated viral vectors, lentiviral vectors, retroviral vectors, and herpes virus vectors. Typically, a vector is capable of replication in a microbial host, for instance, a fungus, such as S. cerevisiae, or a prokaryotic bacterium, such as E. coli, or a cultured cell such as a mammalian or insect cell. In one embodiment, the vector is a plasmid.

Selection of a vector depends upon a variety of desired characteristics in the resulting construct, such as a selection marker, vector replication rate, and the like. In some aspects, suitable host cells for cloning or expressing the vectors herein include eukaryotic cells. Suitable eukaryotic cells include fungi, such as S. cerevisiae and P. pastoris, mammalian cells, and insect cells. In some aspects, suitable host cells for cloning or expressing the vectors herein include prokaryotic cells. Suitable prokaryotic cells include eubacteria, such as gram-negative microbes, for example, E. coli. Vectors may be introduced into a host cell using methods that are known and used routinely by the skilled person. For example, calcium phosphate precipitation, electroporation, heat shock, lipofection, microinjection, and viral-mediated nucleic acid transfer are common methods for introducing nucleic acids into host cells.

An expression vector optionally includes regulatory sequences operably linked to the coding region. The disclosure is not limited by the use of any particular promoter, and a wide variety of promoters are known. Promoters act as regulatory signals that bind RNA polymerase in a cell to initiate transcription of a downstream (3′ direction) coding region. The promoter used may be a constitutive or an inducible promoter. It may be, but need not be, heterologous with respect to the host cell.

An expression vector may optionally include a ribosome binding site and a start site (e.g., the codon ATG) to initiate translation of the transcribed message to produce the protein. It may also include a termination sequence to end translation. A termination sequence is typically a codon for which there exists no corresponding aminoacetyl-tRNA, thus ending protein synthesis. The polynucleotide used to transform the host cell may optionally further include a transcription termination sequence.

A vector introduced into a host cell optionally includes one or more marker sequences, which typically encode a molecule that inactivates or otherwise detects or is detected by a compound in the growth medium. For example, the inclusion of a marker sequence may render the transformed cell resistant to an antibiotic, or it may confer compound-specific metabolism on the transformed cell. Examples of a marker sequence are sequences that confer resistance to kanamycin, ampicillin, chloramphenicol, tetracycline, and neomycin.

A N protein of the present disclosure may be produced using recombinant DNA techniques, such as an expression vector present in a eukaryotic or prokaryotic cell. Such methods are routine and known in the art. The protein may also be synthesized in vitro, e.g., by solid phase peptide synthetic methods. The solid phase peptide synthetic methods are routine and known in the art. A protein produced using recombinant techniques or by solid phase peptide synthetic methods may be further purified.

N protein binds RNA and in natural environments N protein is bound to RNA. The interaction is non-specific but strong, and expression of N protein in a host cell results in expressed N protein that is bound to RNA. As described herein, the inventors have found that use of N protein in a system for detecting anti-N protein antibody results in high background, but use of N protein substantially free of RNA in the system greatly reduces background and results in increased sensitivity. Accordingly, in one embodiment N protein of the present disclosure is substantially free of RNA. In one embodiment, whether N protein is substantially free of RNA can be determined by measuring the optical density of a mixture of N protein at 260 nm and 280 nm and determining the 260/280 ratio. An ideal 260/280 ratio for purified proteins is 0.6, which indicates the amount of contaminating nucleic acid is undetectable. Higher ratios indicate the contamination of isolated proteins with nucleic acid including RNA.

The inventors have prior experience with removing RNA from recombinantly produced RNA-binding Protein Activator of PKR (PACT). Efforts at RNA removal included use of heparin column, low pH treatment, high concentration of RNase A and RNase III treatment, and introducing mutations into the consensus RNA binding residues of two domains of PACT that specifically bind RNA; however, none of these worked well in removing RNA from the recombinantly purified PACT protein. The inventors determined that denaturation of the SARS-CoV-2 N protein followed by isolation and then renaturation resulted in N protein with RNA present at a level that was undetectable. Thus, a N protein substantially free of RNA does not naturally exist and is not a natural form of the protein. Exemplary conditions for isolation of N protein substantially free of RNA are disclosed in Example 1.

In one embodiment, N protein substantially free of RNA can be obtained by suspending a sample that contains N protein, for instance a cell that contains recombinantly expressed N protein, in a denaturing solution, and then lysing the cells. The denaturing solution can be essentially any composition that denatures proteins, and in one embodiment includes urea. The concentration of the denaturant, such as urea, can depend upon the concentration of N protein and other components and can be easily determined by the skilled person. In one embodiment, the concentration of urea is at least 2 M, at least 3 M, at least 4 M, at least 5 M, at least 6 M, at least 7 M, or at least 8 M. In one embodiment, the concentration of urea is no greater than 9 M, no greater than 8 M, no greater than 7 M, no greater than 6 M, no greater than 5 M, no greater than 4 M, or no greater than 3 M. In one embodiment, the concentration of urea can be from 2 M to 9 M, or any value between 2 M and 9 M. The denatured N protein can be separated from other proteins present, and then exposed to a renaturing solution. An example of a renaturing solution is a composition that is similar or identical to the denaturing solution but not containing the denaturant. The resulting N protein can be further processed using standard methods such as concentrating and/or dialysis and the amount of contaminating nucleic acid can be determined.

Antibody may be produced using N protein of the present disclosure. The antibody may be polyclonal or monoclonal. Laboratory methods for producing, characterizing, and optionally isolating polyclonal and monoclonal antibodies are known in the art (see, for instance, Harlow E. et al. Antibodies: A laboratory manual Cold Spring Harbor Laboratory Press, Cold Spring Harbor, 1988). For instance, a protein of the present disclosure may be administered to a subject, preferably a mammal, in an amount effective to cause the production of antibody specific for the administered protein. Optionally, a protein may be mixed with an adjuvant, for instance Freund's incomplete adjuvant, to stimulate the production of antibodies upon administration. Whether an antibody of the present disclosure specifically binds to a protein described herein may be determined using methods known in the art.

The present disclosure also includes genetically modified microbes that have a polynucleotide encoding a N protein of the present disclosure. Compared to a control microbe that is not genetically modified according to the present disclosure, a genetically modified microbe exhibits production of a N protein. A polynucleotide encoding a N protein may be present in the microbe as a vector or integrated into a chromosome. Polynucleotides encoding a N protein of the present disclosure are described herein.

As described in Example 3, the inventors observed a surprisingly high level of N protein expression when a coding region optimized for expression in human cells was expressed in the prokaryotic cell E. coli. It was expected that N protein expression from the human-optimized coding region in E. coli would be lower than N protein expression from the E. coli-optimized coding region in E. coli; however, there was at least 20-fold higher expression from the human-optimized coding region in E. coli. Accordingly, in one embodiment, the genetically modified cell is a prokaryotic cell, including a member of the family Enterobacteriaceae, such as E. coli, that includes a N protein coding region that is optimized for expression in human cells. An example of such a coding region is SEQ ID NO:2, or one having sequence similarity of at least 80% identity to SEQ ID NO:2 that encodes the N protein of SEQ ID NO:1. Another example of a coding region is one that is optimized for expression in human cells and encodes the N protein of SEQ ID NO:1 or a N protein having sequence similarity of at least 80% identity to SEQ ID NO:1.

The present disclosure also includes methods of making N protein, where the method includes culturing a cell that contains a coding region encoding the N protein. In one embodiment, the cell is a prokaryotic cell, for instance a member of the family Enterobacteriaceae, such as E. coli. In one embodiment, the coding region can be SEQ ID NO:2, or one having sequence similarity of at least 80% identity to SEQ ID NO:2 that encodes the N protein of SEQ ID NO:1, where the coding region is optimized for expression in human cells. In another embodiment, the coding region is optimized for expression in human cells and encodes the N protein of SEQ ID NO:1 or a N protein having sequence similarity of at least 80% identity to SEQ ID NO:1. In one embodiment, the coding region can be SEQ ID NO:3, or one having sequence similarity of at least 80% identity to SEQ ID NO:3 that encodes the N protein of SEQ ID NO:1, where the coding region is optimized for expression in a prokaryotic cell, for instance a member of the family Enterobacteriaceae, such as E. coli. In one embodiment, expression of N protein by a genetically modified prokaryotic cell, for instance a member of the family Enterobacteriaceae, such as E. coli, that contains a coding region optimized for expression in a human cell produces more N protein than a control genetically modified cell that contains a N protein coding region optimized for expression in a prokaryotic cell, e.g., a member of the family Enterobacteriaceae, such as E. coli. The difference in expression of N protein can be at least 5-fold, at least 10-fold, at least 15-fold, or at least 20-fold greater in the genetically modified cell containing the coding region optimized for expression in a human cell when compared to the control cell.

The present disclosure provides compositions. In one embodiment, a composition includes a N protein as described herein. The protein can be isolated or purified. A composition can optionally further include a pharmaceutically acceptable carrier. “Pharmaceutically acceptable” refers to a diluent, carrier, excipient, salt, etc., that is compatible with the other ingredients of the composition, and not deleterious to the recipient thereof. Exemplary pharmaceutically acceptable carriers include buffer solutions and generally exclude blood products such as, for example, whole blood and/or plasma. The compositions as described herein may be formulated in pharmaceutical preparations in a variety of forms adapted to the chosen route of administration, including routes suitable for stimulating an immune response to the N protein. Thus, a composition as described herein can be administered via known routes including, for example, oral; parenteral including intradermal, transcutaneous and subcutaneous, intramuscular, intravenous, intraperitoneal, etc. and topically, such as, intranasal, intrapulmonary, intramammary, intravaginal, intrauterine, intradermal, transcutaneous and rectally, etc. It is foreseen that a composition can be administered to a mucosal surface, such as by administration to the nasal or respiratory mucosa (e.g., via a spray or aerosol), in order to stimulate mucosal immunity, such as production of secretory IgA antibodies, throughout a subject's body.

A composition of the present disclosure is administered in an amount sufficient to provide an immunological response to a N protein described herein. The amount administered will vary depending on various factors including, but not limited to, physical condition and age of the subject, and the route of administration. Thus, the absolute amount of N protein included in a given unit dosage form can vary, and depends upon factors such as the species, age, weight and physical condition of the subject, as well as the method of administration. Such factors can be determined by one skilled in the art.

A composition including a pharmaceutically acceptable carrier can also include an adjuvant. An “adjuvant” refers to an agent that can act in a nonspecific manner to enhance an immune response to a particular antigen, thus potentially reducing the quantity of antigen necessary in any given immunizing composition, and/or the frequency of injection necessary in order to generate an adequate immune response to the antigen of interest.

Also provided are methods of using the proteins described herein. In one embodiment, N protein described herein is useful as a reagent in immunoassay to detect antibodies to the protein. Accordingly, the present disclosure provides methods for detecting antibody that specifically binds N protein. These methods are useful in, for instance, determining whether a sample includes antibody the specifically binds N protein. In one embodiment, the method can be used to detect whether a subject has antibody that specifically binds N protein, and diagnosing whether a subject may have, or have had, an infection caused by SARS-CoV-2. In one embodiment, such diagnostic systems are in kit form. The methods include contacting an antibody with a preparation that includes N protein to result in a mixture. In one embodiment, the antibody is present in a biological sample. The method further includes incubating the mixture under conditions to allow the antibody to specifically bind a protein to form an antibody:antigen complex. As used herein, the term “antibody:antigen complex” refers to the complex that results when an antibody specifically binds to a protein. The preparation that includes N protein may also include reagents, for instance a buffer, that provide conditions appropriate for the formation of the antibody:antigen complex. The antibody:antigen complex is then detected.

The methods for detecting the presence of antibodies that specifically bind N protein can be used in various immunoassay formats that have been used to detect antibody. Suitable immunoassay formats include enzyme-linked immunosorbent assay (ELISA), enzyme immunodot assay, agglutination assay, antibody-peptide-antibody sandwich assay, peptide-antibody-peptide sandwich assay, or other well-known immunoassay formats to the ordinarily skilled artisan. These immunoassay formats and procedures have been described in many standard immunology manuals and texts, see for example, by Harlow et al., (1988) Antibodies: A Laboratory Manual, Cold Spring Harbor Laboratory Press, Cold Spring Harbor, N.Y. In one embodiment, the immunoassay format is ELISA. In the ELISA format, a solid phase is coated with the above-identified peptide compositions of the present disclosure. An example of a solid phase is a 96 well plate. Other ELISA formats and procedures are well known in the art.

A sample can be a biological sample. Examples of biological samples include, but are not limited to, blood including serum and/or plasma, and secretions of respiratory tract including sputum. The method can include detecting in a biological sample of a subject the presence or absence of antibody that specifically binds SARS-CoV-2 N protein. The biological sample can be obtained from any subject that might have been exposed to, or infected by, SARS-CoV-2. Examples of subjects include, but are not limited to, a natural host of SARS-CoV-2, an ungulate, a companion animal, or a human. Examples of ungulates include animals that are bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou and reindeer), and Bison (including, for instance, buffalo). Other subjects include an avian (including, for instance, turkeys and chickens), a feline (e.g., domesticated cat and tiger), and a canine (e.g., domesticated dog) animal, a bat, and a pangolin. Examples of companion animals include dogs and cats. In one embodiment, a subject is a mouse. In one embodiment, a subject is a ferret.

In one embodiment, the antibody in the sample is IgG. An agent that binds the antibody in the sample can be used to determine if antibody-antigen complexes are present in the immunoassay. In one embodiment, the agent includes a secondary antibody can be used to detect the complex. The agent can be labeled with any suitable reporter molecule including, but not limited to, a heavy metal, a fluorescent molecule, a luminescent molecule, a radioactive tag, or an enzymatic tag. An example of an enzymatic tag is horseradish peroxidase.

In one embodiment, the method of the present disclosure includes administering to a subject an effective amount of a composition that includes at least one protein described herein. The composition may further include a pharmaceutically acceptable carrier. As used herein, an “effective amount” of a composition of the present disclosure is the amount able to elicit the desired response in the recipient. Examples of subjects include, but are not limited to, a natural host of SARS-CoV-2, an ungulate, a companion animal, or a human. Examples of ungulates include animals that are bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou and reindeer), and Bison (including, for instance, buffalo). Other subjects include an avian (including, for instance, turkeys and chickens), a feline (e.g., domesticated cat and tiger), and a canine (e.g., domesticated dog) animal, a bat, and a pangolin. Examples of companion animals include dogs and cats. In one embodiment, a subject is a mouse. In one embodiment, a subject is a ferret. In some aspects, the methods may further include additional administrations (e.g., one or more booster administrations) of the composition to the animal to enhance or stimulate a secondary immune response. A booster can be administered at a time after the first administration, for instance, 1 to 8 weeks, preferably 2 to 4 weeks, after the first administration of the composition. Subsequent boosters can be administered one, two, three, four, or more times annually.

In one aspect, the disclosure is directed to methods for making antibody to N protein described herein, for instance, by inducing the production of antibody in a subject. The method includes administering to a subject an effective amount of a composition that includes N protein. An “effective amount” of N protein is an amount effective to result in the production of antibody in the animal. Methods for determining whether a subject has produced antibody that specifically bind proteins present in a composition of the present disclosure can be determined as described herein.

In one aspect the disclosure is also directed to treating an infection in a subject caused by SARS-CoV-2a. Methods for determining whether an infection is caused by SARS-CoV-2 are routine and known in the art. In another aspect, the present disclosure is directed to methods for treating one or more signs or symptoms of certain conditions in animals that may be caused by infection by SARS-CoV-2. The method of treating an infection, a sign, or a symptom includes administering to a subject an effective amount of a composition that includes N protein. Treatment of an infection and treatment of signs or symptoms of these conditions can be prophylactic or, alternatively, can be initiated after the infection or development of a condition described herein. Treatment that is prophylactic, for instance, initiated before a subject is infected or manifests signs or symptoms of a condition caused by SARS-CoV-2, is referred to herein as treatment of a subject that is “at risk” of infection or developing the condition. Typically, a subject “at risk” of infection or developing a condition is a subject likely to be exposed to SARS-CoV-2. Accordingly, administration of a composition can be performed before, during, or after the occurrence of the infection or conditions described herein. Treatment initiated after the infection or development of a condition may result in decreasing the severity of the infection, signs, or symptoms of one of the conditions, including completely removing the infection, signs, or symptoms. In treating an infection, a sign, or a symptom, an “effective amount” is an amount effective to prevent the infection or manifestation of signs or symptoms of a condition, decrease the severity of the infection or the signs or symptoms of a condition, and/or completely remove the infection, signs, or symptoms.

The present disclosure also provides a kit for detecting antibody that specifically binds proteins of the present disclosure. The kit includes N protein in a suitable packaging material in an amount sufficient for at least one assay. Optionally, other reagents such as buffers and solutions needed to practice the disclosure are also included. Instructions for use of the packaged proteins are also typically included.

As used herein, the phrase “packaging material” refers to one or more physical structures used to house the contents of the kit. The packaging material is constructed by known methods, preferably to provide a sterile, contaminant-free environment. The packaging material has a label which indicates that the proteins can be used for detecting antibodies induced by SARS-CoV-2. In addition, the packaging material contains instructions indicating how the materials within the kit are employed to detect such antibodies. As used herein, the term “package” refers to a solid matrix or material such as glass, plastic, paper, foil, and the like, capable of holding within fixed limits the proteins. Thus, for example, a package can be a microtiter plate well to which microgram quantities of proteins have been affixed. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

Biochemical Characterization of Recombinant SARS-CoV-2 Nucleocapsid Protein and Serological Assay Development

Abstract. SARS-CoV-2 nucleoprotein (N) binds single-stranded viral RNA genome to form helical ribonucleoprotein complex that is packaged into virion particles. N is relatively conserved among coronaviruses and consists of two structural domains, N-terminal domain (NTD) and C-terminal domain (CTD), that are flanked by three disorganized regions. N is highly immunogenic and has been widely used to develop serological assays as diagnostic tools for COVID-19 infection, although there is a concern that the natural propensity of N to associate with RNA might compromise the assay's specificity. We have expressed and purified from bacterial cells two recombinant forms of SARS-CoV-2 N, one from the soluble fraction of bacterial cell lysates that is strongly associated with bacterial RNAs and the other that is completely devoid of RNAs. We have shown that both forms of N can be used to develop enzyme-linked immunosorbent assays (ELISAs) for specific detection of human and mouse anti-N monoclonal antibodies (mAb) as well as feline SARS-CoV-2 seropositive serum samples, but that the RNA-free form of N exhibits a slightly higher level of sensitivity than the RNA-bound form to react to anti-N mouse mAb. Using electrophoretic mobility shift assay (EMSA), we have also shown that N preferentially binds ssRNA in a sequence-independent manner and that both NTD and CTD of N contribute to RNA-binding activity. Collectively, our study describes the biochemical characterization of SARS-CoV-2 N and its utility in the development of serological assays to detect SARS-CoV-2 infection in humans and companion animals.

Introduction. Current COVID-19 pandemic is caused by SARS-CoV-2, a novel severe acute respiratory syndrome (SARS)-related coronavirus (lineage 2B, genus Betacoronavirus) with 80% identity to SARS-CoV and highest similarity to a bat CoV^{1,2}. Currently there are no effective antiviral compounds against SARS-CoV-2, and monoclonal antibodies (mAbs) can be used to treat mild to moderate COVID-19 cases but not severe disease³. FDA has approved several vaccines for emergency use authorization (EUA)⁴, including mRNA-based vaccines by Pfizer/BioNTech and Moderna as well as adenoviral vector-based vaccine by Johnson & Johnson.

Like other coronaviruses (CoVs), SARS-CoV-2 is an enveloped RNA virus with a large (˜30 kb) positive-sense RNA genome, encoding 15 non-structural proteins (NSPs), 4 structural proteins, and multiple accessory proteins that are named successively from ORF3 to ORF10⁵. The structural proteins include spike (S), envelope (E), membrane (M), and nucleocapsid (N). N protein encapsidates viral genomic RNA and forms a helical ribonucleoprotein that is packaged into virion particles^{6,7}. In addition to viral RNA packaging and viral particle formation⁸, N has been shown to participate in subgenomic mRNA transcription by recruiting the human RNA helicase DDX1 to facilitate RNA template readthrough⁹.

N is one of the most abundantly expressed structural proteins in the CoV life cycle and is relatively conserved among CoVs¹⁰. N protein of SARS-CoV-2 has over 90% sequence similarity with SARS and ˜60% similarity with MERS, another human CoV pathogen within the betacoronavirus genus. All CoV N proteins share similar domain architecture¹¹, consisting of two conserved structural domains, the N-terminal domain (NTD) and C-terminal domain (CTD), which are flanked by three disordered regions¹². NTD, from aa 40 to 180 of SARS-CoV-2 N, is the RNA-binding domain, while CTD, from aa 247 to 364 of SARS-CoV-2 N, is the protein dimerization domain and binds RNA¹³⁻¹⁷. These two structural domains do not interact with each other. Three disordered regions of SARS-CoV-2 N are located around aa 1-40, aa 180-247, and aa 365-418. The functions of the disordered regions have not been clearly defined, though they are highly enriched in basic residues and can also bind RNA¹¹. Although structures of NTD and CTD have been determined¹³⁻¹⁵, no structure of the full-length N or any of its domains in complex with nucleic acids is available.

N protein is the major target of antibody responses after viral infection¹⁸, and has been used in the development of major serological diagnostic tools for COVID-19 infection^{19,20}. Due to its high level of immunogenicity and potential to induce cross reactive immune responses¹⁰, N protein is considered as another major target for vaccine development, although experimental evidence for its protective immunity is still lacking.

In this study, we expressed and purified from bacteria two different forms of the recombinant SARS-CoV-2 N; a soluble form that is strongly associated with bacterial RNAs and the other that is completely free of RNAs. We used these two forms of N to develop serological assays and compared their specificity and sensitivity against a known human monoclonal antibody (mAb) of SARS-CoV-2 and used the more sensitive assay to survey cat sera collected from the Veterinary Medical Center at the University of Minnesota for evidence of potential exposure to SARS-CoV-2. Using a biochemical assay, we also found that recombinant N protein preferentially binds to ssRNA, which is consistent with its expected role in encapsidating viral ssRNA genome during the authentic viral life cycle.

Materials and Methods

Plasmids. The full-length SARS-CoV-2 N gene was synthesized by Twist Biosciences (San Francisco, Calif., USA) and cloned into the bacterial protein expression vector pRSF-Duet1 with an N-terminal His tag and a C-terminal Strep tag to generate the plasmid called pRSV-CoV2-N. The fragments encoding the N-terminal domain (NTD) (44-180 aa) and C-terminal domain (CTD) (247 to 364 aa) of N protein were individually cloned into the bacterial protein expression vectors to generate the pRSV-CoV2-N-NTD and pRSV-CoV2-N-CTD plasmids.

Expression and purification of recombinant N protein from the soluble fraction of bacterial cell lysates. BL21(DE3) bacterial cells that were separately transformed with the aforementioned N protein expression plasmids were grown at 37° C. and pre-chilled at 4° C. before the addition of isopropyl β-d-1-thiogalactopyranoside (IPTG) (Sigma-Aldrich, St. Louis, Mo., USA) at a final concentration of 0.1 mM, and cultured at 16° C. for 18 h. Bacterial cells were collected and resuspended in buffer (20 mM Na₂HPO₄, pH 7.4, 500 mM NaCl), and lysed by sonication. The supernatants were collected after centrifugation at 23,000 g for 60 min, filtered, and applied onto HisTrap HP column (GE healthcare, Chicago, Ill., USA) with the AKTApure chromatography system. After washing with at least 20 column volumes of the wash buffer (20 mM Na₂HPO₄, pH 7.4, 500 mM NaCl, 50 mM imidazole), the protein(s) were eluted with 10 column volumes of the elution buffer (20 mM Na₂HPO₄, pH 7.4, 500 mM NaCl, 500 mM imidazole). The fractions containing the recombinant N protein(s) were pooled and concentrated using the Amicon Ultra-15 centrifugal filter unit (Millipore, Burlington, Mass., USA; cat. #UFC900308). The protein(s) were analyzed for OD260/280 ratio by Nanodrop and quantified by the Bradford assay (BioRad, Hercules, Calif., USA).

Purification of recombinant N protein without associated bacterial RNAs. BL21(DE3) bacterial cells that were separately transformed with the aforementioned N expression plasmids were grown at 37° C. and induced overnight with 1 mM IPTG. The bacterial cells were collected and resuspended in the denaturing buffer (20 mM Na₂HPO₄, pH 7.4, 500 mM NaCl, and 6 M urea), and lysed by sonication. The supernatants were collected after centrifugation at 25,000 g for 60 min, filtered and applied onto the Histrap HP column (GE healthcare) with the AKTApure chromatography system. The column was washed with the same denaturing buffer, followed by a renaturing buffer (20 mM Na₂HPO₄, pH 7.4, 500 mM NaCl) and the HisTrap wash buffer (20 mM Na₂HPO₄, pH 7.4, 500 mM NaCl, 50 mM imidazole). The protein(s) were then eluted with 10 column volumes of the HisTrap elution buffer (20 mM Na₂HPO₄, pH 7.4, 500 mM NaCl, and 500 mM imidazole). Fractions containing the recombinant N protein(s) were pooled and dialyzed against PBS buffer overnight. The protein(s) were analyzed for OD260/280 ratio by Nanodrop and quantified by the Bradford assay (BioRad).

Electrophoretic mobility shift assay (EMSA). Single-stranded (ss) RNAs were generated from a PCR product that contained 5′ T7 promoter and the first 218 nucleotides (nt) of the cDNA sequence of the Lassa virus L polymerase gene, using the MEGAshortscript™ T7 Transcription Kit (Thermo Fisher Scientific, Waltham, Mass., USA) following the manufacturer's instructions. The RNA products were treated with DNase I (New England Biolabs, Ipswich, Mass.), extracted with phenol/chloroform, precipitated with ethanol in the presence of 0.3 M sodium acetate, and dissolved in nuclease-free water (Thermo Fisher Scientific). To generate double-stranded (ds) RNAs, two ssRNAs were independently transcribed from the respective PCR products that contain the reverse complementary DNA sequences of the 218-nt Lassa virus L gene fragment under the T7 promoter, following the methods as described above. The two reverse-complementary ssRNAs were then mixed at an equal concentration of 20 uM in annealing buffer (60 mM KCl, 10 mM HEPES-pH 7.5, 0.2 mM MgCl₂), heated at 95° C. for 2 minutes, and slowly cooled down to 25° C. at a rate of 1° C. per min.

To conduct EMSA, 1 ug of RNA templates (ssRNA or dsRNA) was incubated with increasing concentrations (from 0 to 10 uM) of the purified N protein(s) in a binding buffer (20 mM HEPES, 100 mM NaCl, 10% Glycerol) at room temperature for 30 mins. The samples were separated in a 1% agarose gel with ethidium bromide using 1× Tris-acetic acid-EDTA (1×TAE) buffer and imaged by the myECL imager (Thermo Fisher Scientific).

Evaluation of the recombinant SARS-CoV-2 N protein using reagents of a commercial human IgG/IgM ELISA kit. The purified recombinant SARS-CoV-2 N protein was evaluated using reagents in a commercial COVID-19 N-based human IgG/IgM ELISA kit (MyBioSource, San Diego, Calif., USA; cat. no. MBS3809905). Specifically, a 96-well Maxisorp ELISA plate (Thermo Fisher Scientific) was coated with the recombinant N with associated bacterial RNA, named N(+RNA), or without the associated RNA, named N(−RNA) at 50 ng, 100 ng, and 200 ng per well. The plate was incubated at 37° C. for 1 h, washed three times with phosphate buffer saline containing 0.05% Tween-20 (i.e., PBST buffer), blocked with 5% non-fat milk in PBST buffer at 37° C. for 1 h, and washed three times with PBST buffer. The positive and negative controls supplied in the kit were then added to the respective wells. ELISA was conducted following the manufacturer's instructions.

Comparison of N(+RNA) and N(−RNA) in the N-based ELISA with serially diluted anti-N monoclonal antibody (mAb). A 96-well Maxisorp plate (Thermo Fisher Scientific, cat. no. 437111) was coated with 100 ng of the purified N(+RNA) or N(−RNA) protein overnight at 4° C., blocked with 5% non-fat milk in PBST buffer at 37° C. for 1 h, and washed with PBST buffer as described above. PBS or anti-SARS-CoV N monoclonal antibody (obtained from L. Martinez-Sobrido, Texas Biomedical Research Institute) in serial two-fold dilutions was added to N(+RNA) and N(−RNA)-coated wells, each in triplicates, and incubated for 1 h at 37° C. Following the washing step with PBST, HRP-conjugated goat anti-mouse IgG polyclonal antibody (R&D Systems, cat. no. HAF007) at a 1:1000 dilution was added and incubated for 45 min at 37° C. Following the addition of substrate ABTS (Millipore Sigma, cat. no. A9941) and stop solution (3N HCl), the samples were subjected to absorbance reading at 450 nm wavelength (OD₄₅₀) using the Synergy2 multiplate reader (BioTek, Winooski, Vt., USA). Normalized OD₄₅₀ value of each sample in the 96-well plate was calculated by subtracting OD₄₅₀ value of the un-coated well from OD₄₅₀ value of the corresponding coated well. The cut-off OD₄₅₀ value was set as the mean plus three standard deviations of the OD₄₅₀ values obtained from the negative controls. N-specific IgG endpoint titer was determined as the highest dilution to give an OD₄₅₀ value exceeding the cut-off OD₄₅₀ value, which was set as the mean plus four standard deviations of OD₄₅₀ value obtained from the negative samples that contain the secondary antibody alone.

Home-made N-based cat IgG ELISA. A similar ELISA method as described above was used to evaluate thirty one (31) cat serum samples that were previously determined to be either positive or negative for anti-N antibody seroprevalence through various assays (Example 2). Briefly, a 96-well Maxisorp plate was either un-coated or coated with 100 ng of the purified N(+RNA) or N(−RNA) protein, in duplicates, incubated overnight at 4° C., and blocked with 5% non-fat milk in PBST buffer for 1 h at 37° C. Heat-inactivated cat sera were diluted to 1:50 with 1% non-fat milk in PBST, added equally to both uncoated and coated wells, and incubated for 1 h at 37° C. Following washing with PBST, HRP-conjugated goat anti-cat IgG polyclonal antibody (Rockland Immunochemical, Gillbertsville, Pa., USA; cat. no. RL602-1302) was added at a 1:1000 dilution and incubated for 45 min at 37° C. Following the addition of substrate ABTS and stop solution (3N HCl), the absorbance at 450 nm wavelength (OD₄₅₀) was measured using the Synergy2 multiplate reader (BioTek). Normalized OD₄₅₀ value of each sample was calculated by subtracting OD₄₅₀ value of un-coated well from OD₄₅₀ value of coated well. The cut-off OD₄₅₀ value was set as the mean plus three standard deviations of the OD₄₅₀ values obtained from the negative samples.

Cat serum samples. De-identified cat serum samples were collected for routine diagnostics of cats for illness, wellness or chronic disease monitoring at the Veterinary Medical Center (VMC) of the University of Minnesota, Twin Cities. Serum samples were heat inactivated at 56° C. for 30 min and stored at −20° C. until use.

Results

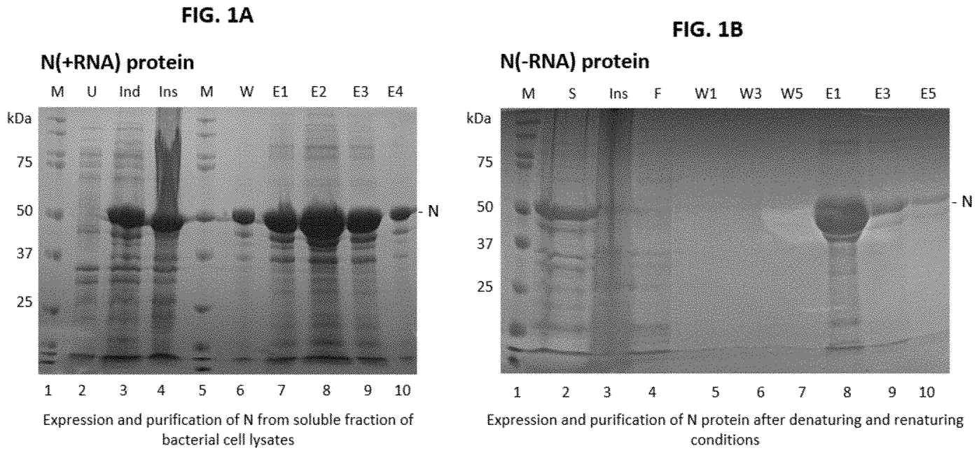

Recombinant SARS-CoV-2 N protein from the soluble fraction of bacterial cell lysates contains bacterial RNAs. We expressed and purified recombinant SARS-CoV-2 N protein from the soluble fractions of bacterial cell lysates by culturing the N-protein plasmid containing bacterial cells at a low temperature (16° C.) and with a relatively low concentration of IPTG (0.1 mM). We analyzed the expression level of the recombinant N protein at each step of the expression and purification process via 12% SDS-PAGE (FIG. 1). When compared to the uninduced condition that produced no SARS-CoV-2 N (FIG. 1, lane 2), recombinant N protein expressed at a relatively high level upon IPTG induction (FIG. 1, lane 3) and remained largely in the insoluble fraction (FIG. 1, lane 4) of the bacterial cell lysates. Recombinant N protein was also present in the soluble fraction, which were purified by the HisTrap affinity column (FIG. 1, lanes 7 to 9). About 2 mg of the recombinant SARS-CoV-2 N protein could be purified from 1 L of the bacteria cell culture. The OD260/280 ratio of the protein sample was 1.84, which suggests that the recombinant SARS-CoV-2 N protein was strongly associated with nucleic acids. RNase A and RNase III treatments of the soluble fraction of the cell lysates prior to the protein purification step did not reduce the presence of nucleic acids in the final protein product, suggesting that nucleic acids are likely integral components of the recombinant SARS-CoV-2 N protein, which is consistent with its function as the nucleocapsid protein that encapsidates viral genomic ssRNAs^{6,7}. For convenience, this form of the SARS-CoV-2 N protein that is intimately associated with bacterial RNAs shall henceforth be referred to as the N(+RNA).

Purification of recombinant SARS-CoV-2 N protein devoid of bacterial RNAs. In order to biochemically characterize the RNA-binding activity of the SARS-CoV-2 N protein, we must develop a method to purify it from bacterial RNAs. Previous studies with SARS-CoV N protein showed that this could be accomplished by chemically inducing protein unfolding by the treatment with urea or guanidine hydrochloride, after which the recombinant protein could then be refolded to its native state and was free of any RNAs^{21,22}. Since the majority of the recombinant SARS-CoV-2 N protein was present in the insoluble fraction (i.e., inclusion body) of the bacterial cell lysates, we decided to use a denaturing buffer that contains a relatively high concentration (6 M) of urea in order to solubilize the inclusion body and to denature the recombinant SARS-CoV-2 N protein (FIG. 1B, lane 2). The unfolded recombinant protein was then applied onto the HisTrap affinity column and washed to remove any protein contaminants (FIG. 1B, lanes 4-7). The recombinant SARS-CoV-2 N protein was then refolded in a renaturing buffer and eluted from the column (FIG. 1B, lanes 8-10). Based on the OD260/280 ratio, the recombinant SARS-CoV-2 N protein was free of nucleic acids. About 150 mg of the RNA-free form of the recombinant SARS-CoV-2 N protein were purified from 1 L of the bacterial cell culture. For convenience, we henceforth refer to it as the N(−RNA).

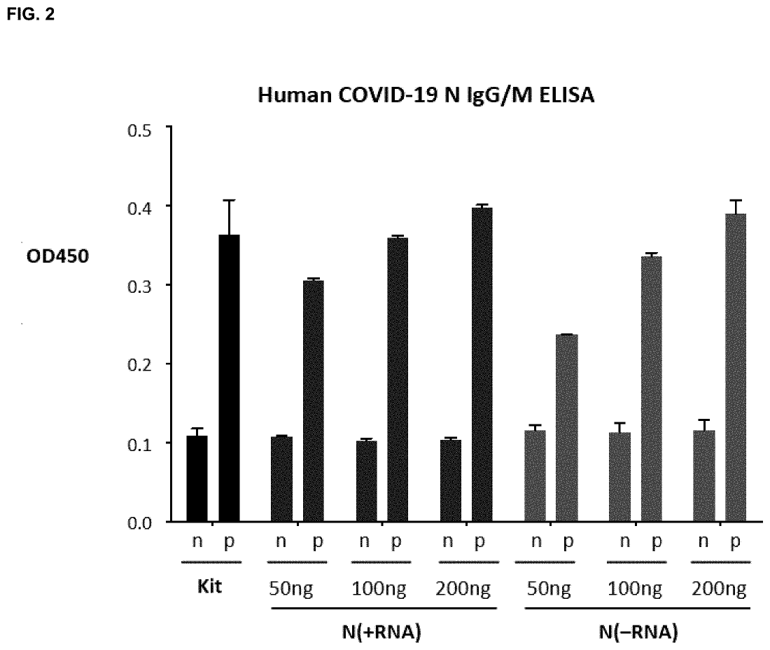

Both N(+RNA) and N(−RNA) proteins can be specifically detected by an anti-N antibody. We next attempted to use N(+RNA) and N(−RNA) proteins to develop an ELISA using reagents in a commercial COVID-19 N-based human IgG/IgM ELISA kit (MyBioSource, cat. no. MBS3809905). Toward this end, we coated ELISA plates with N(+RNA) or N(−RNA) protein at different concentrations (50 ng, 100 ng, and 200 ng per well) and used the positive (human anti-N antibody) and negative controls supplied in the kit in the recommended procedure. Both N(−RNA) and N(+RNA) at different protein concentrations produced consistently low background OD₄₅₀ values with the negative control (n) and significantly high OD₄₅₀ values with the positive control (p) in a dose-dependent manner (FIG. 2). One hundred nanograms (100 ng) of recombinant SARS-CoV-2 N protein, with or without the associated RNAs, produced similar OD₄₅₀ values as those present in the pre-coated plate of the commercial kit. This demonstrates that both forms of the recombinant SARS-CoV-2 N proteins can be specifically recognized by an anti-COVID-19 N antibody.

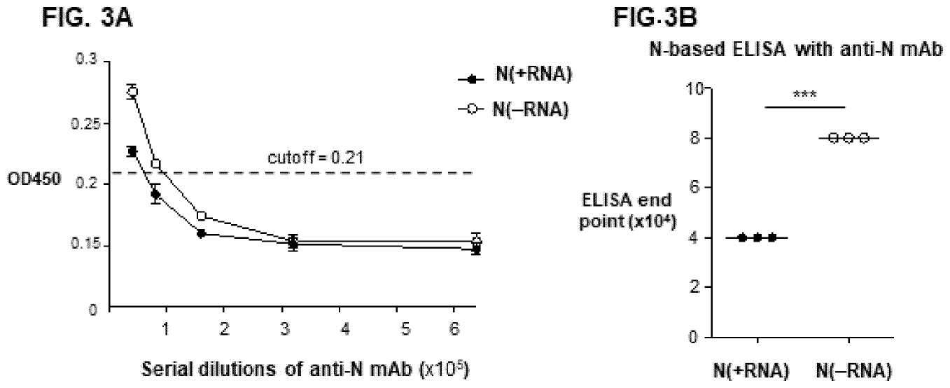

N(−RNA) shows higher sensitivity level than N(+RNA) to detect anti-N antibody by ELISA. To compare the sensitivity levels of the N-based ELISAs that are based on N(−RNA) and N(+RNA), we applied serial dilutions of a mouse anti-SARS-CoV N mAb (as a positive control) into N(−RNA) and N(+RNA)-coated wells, each in triplicates. Shown in FIG. 3A are the OD₄₅₀ values plotted against the mAb dilutions, of which the same dilutions of mAb produced higher OD₄₅₀ values in N(−RNA)-coated wells than in N(+RNA)-coated ones. After calculating the cutoff value at 0.21, the endpoints of the anti-N mAb were 4×10⁴ when N(+RNA) was used and 8×10⁴ when N(−RNA) was used (FIG. 3B), demonstrating that N(−RNA) has a slightly higher level of sensitivity than N(+RNA) in detecting anti-N antibodies.

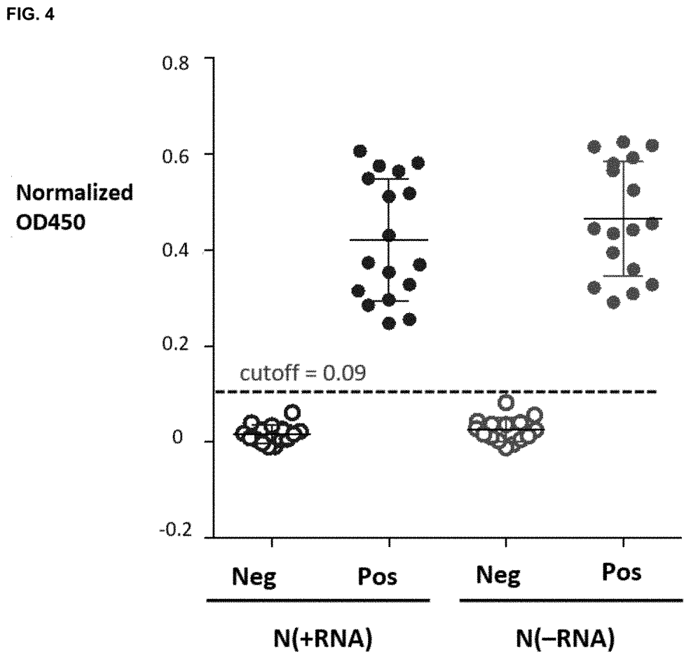

To compare N(−RNA) and N(+RNA) in the application of an N-based IgG ELISA to detect anti-SARS-CoV-2 N antibody in clinical serum samples, we used 31 cat serum samples, including 17 seropositive and 14 seronegative ones, that have previously been confirmed by both the SARS-CoV-2 receptor-binding domain (RBD) and N-based IgG ELISAs as well as in an established COVID-19 neutralization assay (Example 2). Each cat serum sample was added in an equal amount into the wells of the 96-well plate that were either uncoated or coated with N(−RNA) or N(+RNA) in order to carry out the ELISA as described above. The adjusted OD₄₅₀ value was calculated by subtracting the value in uncoated well from that in the respective N-coated well. Both N(+RNA) and N(−RNA)-based IgG ELISA produced consistently low OD₄₅₀ values for all negative samples and significantly higher OD₄₅₀ values than the cutoff value at 0.09 for all positive samples (FIG. 4). The results demonstrate that both N(+RNA) and N(−RNA) proteins can be used to detect anti-N antibodies in clinical serum samples with similar specificity and sensitivity.

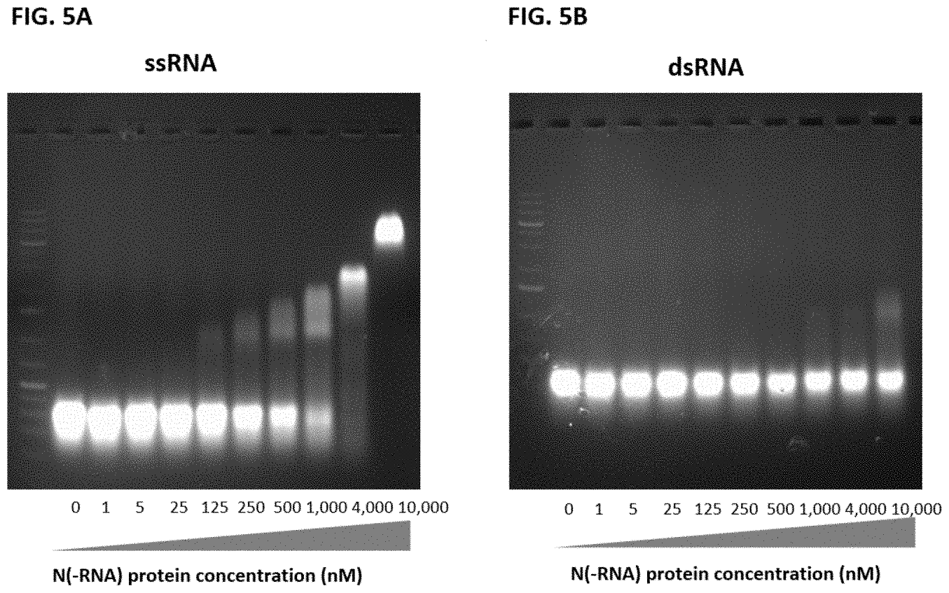

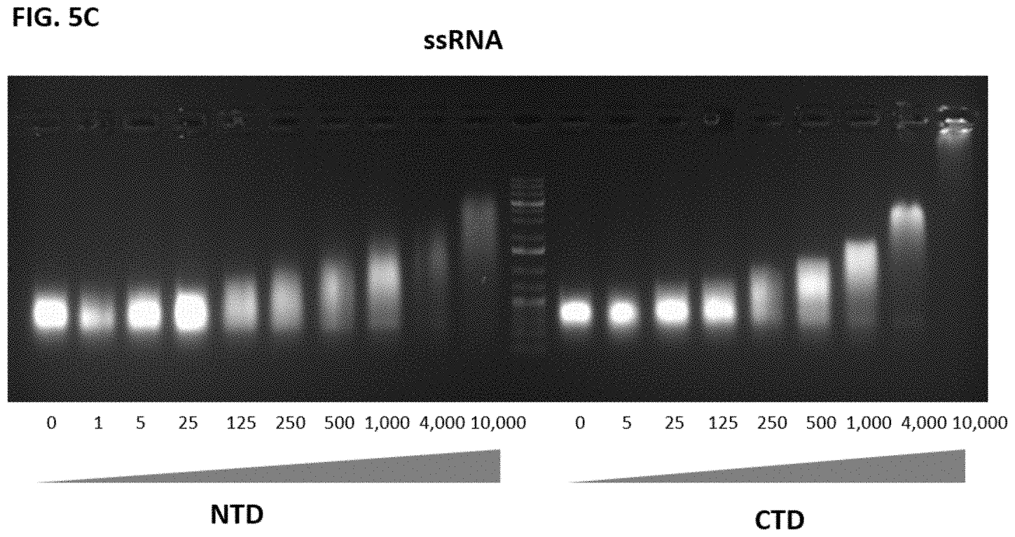

The SARS-CoV-2 N protein preferentially binds ssRNA in vitro. A major function of the CoV N protein is to encapsidate viral ssRNA genome. To determine whether the recombinant N(−RNA) protein is functional in its RNA-binding activity, we conducted EMSA. Single-stranded or double-stranded RNA templates were incubated with increasing concentrations of N(−RNA) and separated on 1% agarose gel. N(−RNA) was shown to bind ssRNA in a concentration-dependent manner (FIG. 5A). The shifted RNA-protein band was first detected at 125 nM of the N(−RNA) protein and increased in its intensity and size along with increasing N(−RNA) protein concentrations, suggesting multimerization of the N(−RNA) with the RNA substrate. At the highest concentration of the N(−RNA) protein (10 uM), however, all ssRNA molecules shifted to form a discrete high molecular-weight band, suggesting that all ssRNAs were bound by the same number of N(−RNA) proteins (FIG. 5A). In contrast, the dsRNA templates did not show any detectable shifted products until the N(−RNA) proteins were at relatively high concentrations, but a significant portion of the dsRNA substrates did not associate with any N(−RNA) protein even at the highest concentration (10 uM) (FIG. 5B), strongly suggesting that the recombinant N(−RNA) protein preferentially binds to ssRNA.

We also expressed and purified NTD and CTD of the N(−RNA) protein and characterized their RNA-binding activities using EMSA. Both NTD and CTD were able to shift ssRNA substrates in a concentration-dependent manner (FIG. 5C), demonstrating that both protein domains have RNA-binding activity, which is consistent with previous findings with CoV N proteins¹³⁻¹⁷.

Discussion

CoV N protein has widely been used for serological diagnosis of viral infection due to its high level of immunogenicity²³, and can also be a good antiviral target due to the essential roles it plays in viral life cycle and its sequence conservation among CoVs^{24,25}. We successfully expressed and purified recombinant SARS-CoV-2 N protein from bacterial cells in two different forms: a soluble N(+RNA) form that can strongly associate with bacterial RNAs and a re-folded N(−RNA) protein that is free of RNAs. We used these two versions of the recombinant SARS-CoV-2 N proteins to conduct biochemical studies and develop serological assays. Both forms of the recombinant SARS-CoV-2 N protein can be used to specifically detect anti-N antibodies (i.e., mAb and antiviral sera) with N(−RNA) form showing a slightly higher level of sensitivity than N(+RNA) (FIG. 3). Given the high yield of the recombinant N(−RNA) produced in bacteria and its high levels of specificity and sensitivity in detecting anti-N antibodies, this N(−RNA) protein is an ideal product for use to develop N-based serological assay to screen for SARS-CoV-2 infections. Given the increasingly high vaccination rates for COVID-19 in the general populations, the N-based COVID-19 serological assays provide a valuable tool to distinguish natural SARS-CoV-2 infection from vaccination, because all current vaccine formulations use the viral spike (S) protein but not N as an antigen. Using the N(−RNA), we set up a new N-based IgG ELISA to screen a relatively small number of the already confirmed household cat's sera as part of a larger study to survey pet cats and dogs admitted to the Veterinary Medical Center at the University of Minnesota that demonstrated a higher seroprevalence of SARS-CoV-2 infection of pet cats than of pet dogs in the early phase of the COVID-19 pandemic in the state of Minnesota (Dileepan et al., manuscript in submission entitled “Seroprevalence of SARS-CoV-2 infection in pet cats and dogs in Minnesota, USA”).

Our biochemical assays suggest that the recombinant SARS-CoV-2 N protein that are expressed and purified in bacteria binds to RNA in a sequence-independent manner and is tightly associated with bacterial RNAs that are strongly resistant to RNase treatments, suggesting that the recombinant SARS-CoV-2 N protects the RNA independently of its sequence and origin. This is consistent with previous findings that CoV N encapsidates viral genomic ssRNA in a sequence-independent manner²⁶.

Previous studies have also shown that CoV N has non-specific binding activity toward nucleic acids, including ssRNA, ssDNA, and dsDNA^{27,28}, although CoV N binding to ssRNA is more specific than to DNA. For example, Tang and colleagues²⁷ showed that the ssRNA-N complexes resolved as a sharp and strong band in the EMSA gel, suggesting that the complexes are uniform in size and are resistant to RNase treatments, while the DNA-N complexes produced a smeary band on the EMSA gel, suggesting that these interactions are less specific²⁷. In our current study, we showed that ssRNA-N interactions appeared to be more specific and stronger than those of the dsRNA-N complexes on the EMSA gels (FIG. 5), suggesting that N binds to ssRNA more specifically than its binding to dsRNA. While we have not determined the kinetics of RNA-N interactions, a recent pre-publication (available in bioRxiv archive) measured the binding affinity of SARS-CoV-2 N to ssRNA and stem-loop RNA (s1RNA) and showed that the binding of SARS-CoV-2 N to s1RNA is in an order of magnitude lower than that to ssRNA²⁹, and is therefore consistent with our current findings.

While the exact mechanism of SARS-CoV-2 N's RNA binding activity is still unclear, a previous study has shown that SARS-CoV N binds to RNA cooperatively and requires multiple regions of the protein, such as NTD, CTD, and its disordered regions¹¹. However, the exact mechanism by which CoV Ns encapsidate viral ssRNA genomes to form nucleocapsid is still poorly understood, partly due to the lack of an atomic structure for the full-length CoV N or any of its predicted domains in complex with nucleic acids. In addition to genome encapsidation, CoV N has recently been shown to participate in the regulation of viral RNA transcription by recognizing viral transcriptional regulatory sequences³⁰, suggesting that, under a certain condition, CoV N may harbor a sequence specific RNA-binding activity.

In summary, we have reported findings on characterizing the biochemical activities of recombinant SARS-CoV-2 N protein with or without the associated RNA by demonstrating its preferential ssRNA-binding activity in vitro, and have developed a method to purify a recombinant SARS-CoV-2 N protein that is free of any RNA and used it to develop serological assay to screen cat's sera for evidence of SARS-CoV-2 infections. We believe that this new serological assay can serve as an important diagnostic tool for the detection of COVID-19 in humans, companion animals, and perhaps other animal species. Future structural and functional characterizations of the recombinant SARS-CoV-2 N are needed in order to facilitate the development of potential SARS-CoV-2 N-targeting antiviral agents.

### CITATIONS FOR EXAMPLE 1

1. Wu F, Zhao S, Yu B, Chen Y-M, Wang W, Song Z-G, Hu Y, Tao Z-W, Tian J-H, Pei Y-Y, et al. A new coronavirus associated with human respiratory disease in China. Nature 2020; 579:265-9.
2. Zhou P, Yang X-L, Wang X-G, Hu B, Zhang L, Zhang W, Si H-R, Zhu Y, Li B, Huang C-L, et al. A pneumonia outbreak associated with a new coronavirus of probable bat origin. Nature 2020; 579:270-3.
3. Commissioner O of the. Coronavirus (COVID-19) Update: FDA Authorizes Monoclonal Antibodies for Treatment of COVID-19 [Internet]. FDA2020 [cited 2021 Jan. 28]; Available from: https://www.fda.gov/news-events/press-announcements/coronavirus-covid-19-update-fda-authorizes-monoclonal-antibodies-treatment-covid-19
4. Commissioner O of the. COVID-19 Vaccines. FDA [Internet] 2021 [cited 2021 Feb. 27]; Available from: https://www.fda.gov/emergency-preparedness-and-response/coronavirus-disease-2019-covid-19/covid-19-vaccines
5. Gordon D E, Jang G M, Bouhaddou M, Xu J, Obernier K, White K M, O'Meara M J, Rezelj V V, Guo J Z, Swaney D L, et al. A SARS-CoV-2 protein interaction map reveals targets for drug repurposing. Nature 2020; 583:459-68.
6. Masters P S. Coronavirus genomic RNA packaging. Virology 2019; 537:198-207.
7. Chang C, Chen C-M M, Chiang M, Hsu Y, Huang T. Transient oligomerization of the SARS-CoV N protein—implication for virus ribonucleoprotein packaging. PLoS One 2013; 8:e65045.
8. Siu Y L, Teoh K T, Lo J, Chan C M, Kien F, Escriou N, Tsao S W, Nicholls J M, Altmeyer R, Peiris J S M, et al. The M, E, and N structural proteins of the severe acute respiratory syndrome coronavirus are required for efficient assembly, trafficking, and release of virus-like particles. J Virol 2008; 82:11318-30.
9. Wu C-H, Chen P-J, Yeh S-H. Nucleocapsid phosphorylation and RNA helicase DDX1 recruitment enables coronavirus transition from discontinuous to continuous transcription. Cell Host Microbe 2014; 16:462-72.
10. Dutta N K, Mazumdar K, Gordy J T. The Nucleocapsid Protein of SARS-CoV-2: a Target for Vaccine Development. J Virol [Internet] 2020; 94. Available from: https://pubmed.ncbi.nlm.nih.gov/32546606/11.
11. Chang C-K, Hsu Y-L, Chang Y-H, Chao F-A, Wu M-C, Huang Y-S, Hu C-K, Huang T-H. Multiple nucleic acid binding sites and intrinsic disorder of severe acute respiratory syndrome coronavirus nucleocapsid protein: implications for ribonucleocapsid protein packaging. J Virol 2009; 83:2255-64.
12. Chang C, Sue S-C, Yu T, Hsieh C-M, Tsai C-K, Chiang Y-C, Lee S, Hsiao H, Wu W-J, Chang W-L, et al. Modular organization of SARS coronavirus nucleocapsid protein. J Biomed Sci 2006; 13:59-72.
13. Peng Y, Du N, Lei Y, Dorje S, Qi J, Luo T, Gao G F, Song H. Structures of the SARS-CoV-2 nucleocapsid and their perspectives for drug design. EMBO J 2020; 39:e105938.
14. Dinesh D C, Chalupska D, Silhan J, Koutna E, Nencka R, Veverka V, Boura E. Structural basis of RNA recognition by the SARS-CoV-2 nucleocapsid phosphoprotein. PLoS Pathog 2020; 16:e1009100.
15. Zinzula L, Basquin J, Bohn S, Beck F, Klumpe S, Pfeifer G, Nagy I, Bracher A, Hartl F U, Baumeister W. High-resolution structure and biophysical characterization of the nucleocapsid phosphoprotein dimerization domain from the Covid-19 severe acute respiratory syndrome coronavirus 2. Biochem Biophys Res Commun [Internet] 2020; Available from: https://pubmed.ncbi.nlm.nih.gov/33039147/0110.000658US01
16. Huang Q, Yu L, Petros A M, Gunasekera A, Liu Z, Xu N, Hajduk P, Mack J, Fesik S W, Olejniczak E T. Structure of the N-terminal RNA-binding domain of the SARS CoV nucleocapsid protein. Biochemistry 2004; 43:6059-63.
17. Yu I-M, Oldham M L, Zhang J, Chen J. Crystal structure of the severe acute respiratory syndrome (SARS) coronavirus nucleocapsid protein dimerization domain reveals evolutionary linkage between corona- and arteriviridae. J Biol Chem 2006; 281:17134-9.
18. Smits V A J, Hernández-Carralero E, Paz-Cabrera M C, Cabrera E, Hernández-Reyes Y, Hernández-Fernaud J R, Gillespie D A, Salido E, Hernandez-Porto M, Freire R. The Nucleocapsid protein triggers the main humoral immune response in COVID-19 patients. Biochem Biophys Res Commun 2021; 543:45-9.
19. Algaissi A, Alfaleh M A, Hala S, Abujamel T S, Alamri S S, Almahboub S A, Alluhaybi K A, Hobani H I, Alsulaiman R M, AlHarbi R H, et al. SARS-CoV-2 S1 and N-based serological assays reveal rapid seroconversion and induction of specific antibody response in COVID-19 patients. Scientific Reports 2020; 10.
20. Ng D L, Goldgof G M, Shy B R, Levine A G, Balcerek J, Bapat S P, Prostko J, Rodgers M, Coller K, Pearce S, et al. SARS-CoV-2 seroprevalence and neutralizing activity in donor and patient blood. Nature Communications 2020; 11.
21. Luo H, Ye F, Sun T, Yue L, Peng S, Chen J, Li G, Du Y, Xie Y, Yang Y, et al. In vitro biochemical and thermodynamic characterization of nucleocapsid protein of SARS. Biophys Chem 2004; 112:15-25.
22. Wang Y, Wu X, Wang Y, Li B, Zhou H, Yuan G, Fu Y, Luo Y. Low stability of nucleocapsid protein in SARS virus. Biochemistry 2004; 43:11103-8.
23. Chew K L, Tan S S, Saw S, Pajarillaga A, Zaine S, Khoo C, Wang W, Tambyah P, Jureen R, Sethi S K. Clinical evaluation of serological IgG antibody response on the Abbott Architect for established SARS-CoV-2 infection. Clin Microbiol Infect 2020; 26:1256.e9-1256.e11.
24. Roh C. A facile inhibitor screening of SARS coronavirus N protein using nanoparticle-based RNA oligonucleotide. Int J Nanomedicine 2012; 7:2173-9.
25. Lin S-M, Lin S-C, Hsu J-N, Chang C-K, Chien C-M, Wang Y-S, Wu H-Y, Jeng U-S, Kehn-Hall K, Hou M-H. Structure-Based Stabilization of Non-native Protein-Protein Interactions of Coronavirus Nucleocapsid Proteins in Antiviral Drug Design. J Med Chem 2020; 63:3131-41.
26. Chang C, Hou M-H, Chang C-F, Hsiao C-D, Huang T. The SARS coronavirus nucleocapsid protein—forms and functions. Antiviral Res 2014; 103:39-50.
27. Tang T-K, Wu M P-J, Chen S-T, Hou M-H, Hong M-H, Pan F-M, Yu H-M, Chen J-H, Yao C-W, Wang A H-J. Biochemical and immunological studies of nucleocapsid proteins of severe acute respiratory syndrome and 229E human coronaviruses. Proteomics 2005; 5:925-37.
28. Zeng W, Liu G, Ma H, Zhao D, Yang Y, Liu M, Mohammed A, Zhao C, Yang Y, Xie J, et al. Biochemical characterization of SARS-CoV-2 nucleocapsid protein. Biochem Biophys Res Commun 2020; 527:618-23.
29. Wu C, Qavi A J, Hachim A, Kavian N, Cole A R, Moyle A B, Wagner N D, Sweeney-Gibbons J, Rohrs H W, Gross M L, et al. Characterization of SARS-CoV-2 N protein reveals multiple functional consequences of the C-terminal domain. bioRxiv [Internet] 2020 [cited 2021 Feb. 26]; Available from: https://www.ncbi.nlm.nih.gov/pmc/articles/PMC7709165/30.
30. Yang M, He S, Chen X, Huang Z, Zhou Z, Zhou Z, Chen Q, Chen S, Kang S. Structural Insight Into the SARS-CoV-2 Nucleocapsid Protein C-Terminal Domain Reveals a Novel Recognition Mechanism for Viral Transcriptional Regulatory Sequences. Front Chem 2020; 8:624765.

### Example 2

Seroprevalence of SARS-CoV-2 Infection in Pet Cats and Dogs in Minnesota, USA

Abstract. The COVID-19 pandemic caused by the coronavirus SARS-CoV-2 is continuing to spread globally. SARS-CoV-2 infections of feline and canine species have also been reported. However, it is not entirely clear to what extent natural SARS-CoV-2 infection of pet dogs and cats is in households. We have developed enzyme-linked immunosorbent assays (ELISAs) using recombinant SARS-CoV-2 nucleocapsid (N) protein and receptor-binding-domain (RBD) of the spike protein, and the SARS-CoV-2 spike-pseudotyped vesicular stomatitis virus (VSV)-based neutralization assay to screen serum samples of 239 pet cats and 510 pet dogs in Minnesota in the early phase of the COVID-19 pandemic from mid-April to early June 2020 for evidence of SARS-CoV-2 exposures. A cut-off value was used to identify the seropositive samples in each experiment. The average seroprevalence of N- and RBD-specific antibodies in pet cats were 8% and 3%, respectively. Among nineteen (19) N-seropositive cat sera, fifteen (15) exhibited neutralizing activity and seven (7) were also RBD-seropositive. The N-based ELISA is also specific and does not cross react with antigens of common feline coronaviruses. In contrast, SARS-CoV-2 antibodies were detected at a very low percentage in pet dogs (˜1%) and were limited to IgG antibodies against SARS-CoV-2 N protein with no neutralizing activities. Our results demonstrate that more pet cats are SARS-CoV-2 seropositive than pet dogs in MN early in the pandemics and that SARS-CoV-2 N-specific IgG antibodies can detect SARS-CoV-2 infections in companion animals with higher levels of specificity and sensitivity than RBD-based ELISA.

Introduction. The new severe acute respiratory syndrome coronavirus 2 (SARS-CoV-2) emerged in late 2019 in Wuhan, China, and is causing the COVID-19 pandemic“ ” that has profound health, social, and economic impacts on a global scale. As of April 2021, there are more than 140 million confirmed COVID-19 cases worldwide and more than 3 million deaths associated with SARS-CoV-2 infection, including about 32 million cases and about 570 million deaths in the US alone.

No effective antiviral compounds are currently available. Monoclonal antibodies (mAbs) can be used to treat mild to moderate COVID-19 patients but not severe disease³. Several vaccines, including mRNA-based vaccines by Pfizer/BioNTech and Moderna as well as adeno viral vector-based vaccine by Johnson & Johnson, have been approved by FDA for emergency use authorization (EUA)⁴. However, the emergence and wide spread of new SARS-CoV-2 variants that can reduce the effectiveness of the current vaccines pose significant threat to combating the pandemic.

SARS-CoV-2 has 80% amino acid sequence identity to SARS-CoV that caused the 2003 SARS outbreak with ˜10% fatality rate. Both are members of the genus Betacoronavirus that also include Middle East respiratory syndrome virus (MERS-CoV), which emerged in 2012 and caused severe respiratory illness with ˜36% fatality rate⁵. All three highly pathogenic CoVs have been reported to originate in bat populations and are believed to transmit to humans through intermediate animal hosts, such as palm civets for SARS-CoV and camels for MERS-CoV, but the potential intermediate host(s) for SARS-CoV-2 remain unknown⁶. SARS-CoV-2, like other coronaviruses, is an enveloped RNA virus with a single-stranded RNA genome of ˜31 kb that encodes 16 non-structural proteins (nsp1-nsp16), 4 structural proteins (spike S, envelope E, membrane M, and nucleocapsid (N)), and several accessory proteins⁷. The spike (S) protein of SARS-CoV-2 protruding from the viral envelope membrane is responsible for viral entry by binding to human angiotensin-converting enzyme 2 (hACE2), the same receptor used by SARS-CoV, through the receptor-binding domain (RBD) on the spike protein and is responsible for mediating the virus-cell membrane fusion⁸⁻¹². SARS-CoV-2 can also infect many animal species¹³. Ferrets, Syrian golden hamsters, domestic cats, cynomolgus macaques, and raccoon dogs have been shown to be highly permissive to SARS-CoV-2 after experimental inoculation and can shed and transmit the virus to co-housed (sentinel) animals¹⁴⁻²¹. Most of the infected animals show no clinical signs and develop none or mild signs of respiratory inflammation, except for Syrian hamsters that can develop severe pathological lung lesions^{15,21}. Dogs can also be experimentally infected with SARS-CoV-2 but do not shed virus, whereas pigs, chickens and ducks are not at all susceptible to experimental SARS-CoV-2 infections^{14,19,22}. Natural infections by SARS-CoV-2 have been reported in pet dogs, cats, zoo tigers and lions that show only mild respiratory signs²³⁻²⁵. In contrast, SARS-CoV-2 has caused widespread and lethal infections in farmed mink in the EU and in the USA^{26,27}. Outbreaks of SARS-CoV-2 infections in both mink and humans on mink farms suggest that the virus can potentially easily cross the natural species barrier between mink and men, raising the concern for zoonosis and reverse zoonosis of SARS-CoV-2 transmission²⁸. The recent isolation of a SARS-CoV-2 mink-associated variant strain with decreased sensitivity to neutralizing antibodies and its infection of more than 200 people so far²⁹ have led to the culling of 17 million farmed mink in Denmark, and have raised a real concern over its impact on the efficacy of the COVID-19 vaccine candidates currently under various stages of development and testing^{30,31}.

There are estimated 76 million pet dogs and 96 million pet cats living in approximately 70% of the U.S. households (²⁴ and references therein). Therefore, these companion animals have very close contacts with humans in close quarters. As companion animals are the potential sources and sentinels of a wide range of infectious diseases, determination of their susceptibility to and prevalence for natural SARS-CoV-2 infections has significant impacts for both animal and human health³²⁻³⁴. We therefore undertook a serological survey of 239 cat sera and 510 dog sera collected in the Veterinary Medical Center (VMC) at the University of Minnesota, Twin Cities in the early period of the COVID-19 epidemic in Minnesota (MN) from mid-April to mid-June of 2020. The IgG antibodies in these pet sera against the SARS-CoV-2 N and spike RBD were measured by indirect ELISAs developed in our laboratory. The levels of neutralizing antibodies (nAbs) in the pet sera against SARS-CoV-2 were also quantified by using a SARS-CoV-2 spike-pseudotyped VSV-based neutralization assay. We have detected higher seroprevalence in pet cats than in pet dogs in MN early in the COVID-19 pandemic. Moreover, SARS-CoV-2 N-based ELISA detected more seropositive samples, which were corroborated by neutralization assay, than RBD-based ELISA.

Materials and Methods:

Animal serum samples. De-identified serum samples were obtained from discarded patient serum samples, which were collected for routine diagnostics of pet cats and dogs for illness, wellness or chronic disease monitoring at the Veterinary Medical Center (VMC) of the University of Minnesota, Twin Cities. Serum samples of 510 pet dogs and 239 pet cats in total were collected between mid-April and mid-June of 2020, heat inactivated at 56° C. for 30 min, and stored at −20° C. until use. No identifiable information about the pets and their owners were made available to the researchers aside from the animal species. The owners of these pets have signed informed consents to allow discarded biological samples (i.e., sera) from their pets to be used in this study.

Production of recombinant nucleocapsid (N) protein and the receptor-binding domain (RBD) of SARS-CoV-2. The full-length SARS-CoV-2 N gene was synthesized by Twist Biosciences (San Francisco, Calif.) and cloned into the bacterial expression vector pRSF-Duet1 with an N-terminal His tag and a C-terminal Strep tag. BL21(DE3) bacterial cells transformed with the plasmid were grown at 37° C. and induced overnight with 1 mM IPTG. The cell pellet was collected by centrifugation at 5,000 g in F15-8x50cy fixed angle rotor for 20 min and resuspended in the denaturing buffer (20 mM Na₂HPO₄, pH 7.4, 500 mM NaCl, and 6 M urea). Cells were lysed by sonication followed by centrifugation at 25,000 g for 60 min. The supernatants were filtered and applied onto Histrap HP column (GE healthcare) with AKTApure chromatography system. The column was washed with the denaturing buffer, followed by the renaturing buffer (20 mM Na₂HPO₄, pH 7.4, 500 mM NaCl) and the HisTrap wash buffer (20 mM Na₂HPO₄, pH 7.4, 500 mM NaCl, 50 mM imidazole) before being eluted with 10× column volume of the HisTrap elution buffer (20 mM Na₂HPO₄, pH 7.4, 500 mM NaCl, and 500 mM imidazole). The fractions containing the recombinant N protein were pooled and dialyzed against PBS buffer overnight. The purified SARS-CoV-2 N protein is free of associated RNA as determined by the OD260/280 ratio and quantified by the Bradford assay. SARS-CoV-2 RBD was expressed from mammalian cells as described previously³⁵ and was a kind gift of Y. Wan and F. Li, University of Minnesota, Twin Cities.

SARS-CoV-2 N- and RBD-based indirect enzyme-linked immunosorbent assays (ELISAs). Maxisorp plates (96 wells) (Thermo Fisher Scientific, Cat #437111) were either un-coated or coated with 100 ng of the purified N or RBD protein in bicarbonate buffer overnight at 4° C. Wells were blocked with 5% non-fat milk in PBS with 0.05% Tween20 (PBST) for 1 h at 37° C. Heat-inactivated sera were diluted to 1:50 with 1% non-fat milk in PBST, added equally to both uncoated and coated wells, and incubated for 1 h at 37° C. Monoclonal antibodies against RBD (CR3022, recombinant human mAb obtained from M. Jenkins, University of Minnesota, Twin cities) or against SARS-CoV N protein (1C7C7L mouse mAb obtained from L. Martinez-Sobrido, Texas Biomedical Research Institute) were included as positive controls. Normal feline (Cat #102643; Jackson Immunoresearch Laboratories Inc) and canine sera (Cat #004-000-001; Jackson Immunoresearch Laboratories Inc) were included as negative controls. Following the wash with PBST, HRP-conjugated goat anti-cat IgG polyclonal antibody (Cat #RL602-1302; Rockland Immunochemical) or goat anti-canine IgG polyclonal antibody (Cat #A18763: Thermo Fisher Scientific) was added at a 1:1000 dilution and incubated for 45 min at 37° C. Following the addition of substrate ABTS (Cat #A9941; Millipore Sigma) and stop solution (3N HCl), the absorbance at 450 nm (OD₄₅₀) was measured using the Synergy2 multiplate reader (BioTek). Normalized OD₄₅₀ of each sample was calculated by subtracting OD₄₅₀ of un-coated well from OD₄₅₀ of coated well. The cut-off value was set as the mean plus three standard deviations of the values obtained from the negative samples. Each serum sample was screened in at least two independent experiments.

ELISA to detect feline infectious peritonitis coronavirus (FIPV). An indirect ELISA was set up to detect FIPV antibodies in the cat sera. The native FIPV antigens (catalog #MBS568874, MyBioSource) were used to coat the plate at 200 ng/well in bicarbonate coating buffer at 4° C. overnight. The plate was blocked with 5% non-fat milk in PBST for 1 h at 37° C. Heat-inactivated cat serum samples were diluted at 1:50 in PBST with 1% non-fat milk and added into both FIPV-coated and un-coated wells. Goat anti-FIPV polyclonal antibody (catalog #MBS560925, MyBioSource) was included as a positive control. The HRP-conjugated goat anti-cat (Cat #RL602-1302; Rockland Immunochemical) and mouse anti-goat (cat #sc-2354, Santa Cruz) secondary antibodies were used at 1:1000 dilution in PBST with 1% non-fat milk. After 10 min-15 min of substrate incubation at 37° C., the stop solution (3N HCl) was added. Normalized OD₄₅₀ was obtained and analyzed as described above.

SARS-CoV-2 spike-pseudotyped vesicular stomatitis virus (VSV)-based neutralization assay. A replication-defective VSV with SARS-CoV-2 spike protein was used to measure the neutralizing antibody titers in the cat's and dog's sera following the procedure as previously described¹⁶. To generate SARS-CoV-2 spike-pseudotyped recombinant VSV, human kidney epithelial (293T) cells on 10 cm² plate were transfected with the plasmid pSARS-CoV-2Δ19 expressing the SARS-CoV-2 spike protein (a kind gift of T. Hatziioannou and P. Bieniasz, Rockefeller University), and 24 h later infected with the seed virus rVSVΔG/Fluc that contains the VSV-G and expresses the firefly luciferase gene (a kind gift of S. Paessler, University of Texas at the Medical Branch), in the presence of I1-Hybridoma (ATCC #CRL-2700) supernatants that contain anti-VSV-G monoclonal antibody. The supernatants were collected 24 h after infection and ultracentrifuged at 27,000 rpm in SW28 rotor for 2 h. The rVSVΔG/Fluc-CoV2-S viral particle pellets were resuspended in PBS, aliquoted, and stored at −80° C. For the neutralization assay, the rVSVΔG/Fluc-CoV2-S pseudotyped virus was incubated with serum samples in serial dilutions for 30 min and applied to 293T/ACE2(B) stable cells (a kind gift of T. Hatziioannou and P. Bieniasz, Rockefeller University) on 96-well plates, in the presence of anti-VSV-G monoclonal antibody. At 16 h post-infection, cells were lysed and the FLuc activity was quantified using the Firefly Luciferase Assay System following the manufacturer's protocol (Promega). The neutralization titer is determined as the serum dilution that reduces the FLuc activity by 50%. To assess whether the pseudotyped viral entry was mediated by SARS-CoV-2 Spike in an hACE2-dependent manner, 293T/ACE2(B) cells were first incubated with increasing concentrations (0, 2.5, 5, 10, 20, and 40 ug/ml) of recombinant hACE2 proteins or bovine serum albumin (BSA) as a negative-control protein, before the addition of the rVSVΔG/Fluc-CoV2-S pseudotyped virus. The FLuc activity was quantified as described above.

Results

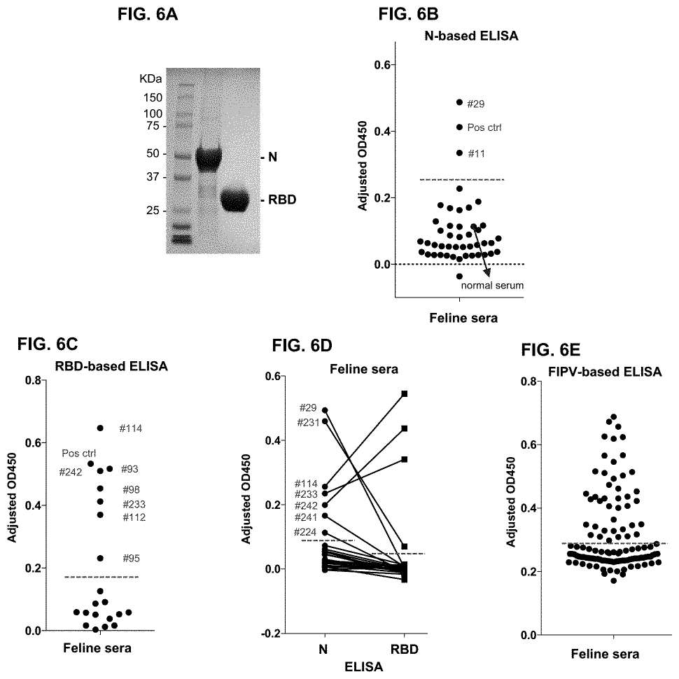

Screening feline serum samples by SARS-CoV-2 N-based ELISA. Nucleocapsid (N) protein encapsidates viral genomic RNA and is one of the most abundant SARS-CoV-2 proteins, whereas spike (S) protein is responsible for mediating virus entry into infected cells⁷. As N and S proteins are known main targets of antibody responses in human COVID-19 patients^{37,38}, current serological assays are mostly based on the N and S or the immunogenic epitopes of S³⁹. Using recombinant full-length SARS-CoV-2 N protein purified from bacteria that is free of any associated RNAs, we have developed an N-based ELISA to detect SARS-CoV-2 IgG antibodies in serum samples of pet cats, which were collected in separate batches at the Veterinary Medical Center of the University of Minnesota, Twin Cities between mid-April to mid-June 2020 (Table 1). Each serum sample was applied onto un-coated and N-coated wells of the 96-well plate to conduct the ELISA as described in the Materials and Method and to obtain OD₄₅₀ measurements. The adjusted OD₄₅₀ values for all serum samples were calculated using the simple formula: OD₄₅₀ (N coated-well)−OD₄₅₀ (un-coated-well). As shown in FIG. 6A, most representative samples, including a normal feline serum as a negative control, have adjusted OD₄₅₀ values that are below 0.25, while three of the samples that include the feline serum sample #11 and #29 and a positive control sample that contains an anti-N mAb showed significantly higher OD₄₅₀ values. Based on these results, the OD₄₅₀ cut-off value used to distinguish positive samples from negative samples was set as the mean OD₄₅₀ values plus three standard deviations of those of the negative samples and shown as a red dashed line (FIG. 6A), which classified feline serum samples #11 and #29 as positive for anti-N antibodies. Out of 239 cat sera screened, 19 samples were deemed to be positive for anti-N antibodies in this N-based ELISA, which yielded an overall seroprevalence of 7.9%. These feline positive serum samples include 2 samples obtained from Apr. 6 to Apr. 27, 2020; 14 samples from May 8 to Jun. 2, 2020; and 3 samples from Jun. 5 to Jun. 12, 2020 (Table 1). There is a noticeable increase of seroprevalence for N-specific IgG antibodies in pet cats since mid-May, 2020 (0-5% seroprevalence before and 11-12% seroprevalence after mid-May) that appears to correspond to an increase in the numbers of COVID-19 in human populations in MN⁴⁰. However, due to the lack of information on the pet owners, it is not clear whether there is a direct correlation between infected pet cats and their owners.

TABLE 1
Feline serum samples screened by SARS-CoV-2 N- and RBD-based ELISAs

| feline | Collection | N ELISA | N sero- | N sero- | RBD sero- | RBD sero- |
|---|---|---|---|---|---|---|
| serum ID | Date | (#) | positive (#) | positive (%) | positive (#) | positive (%) |
| #1-43 | Apr. 16, 2020- | 43 | 2 | 4.7% | 0 | 0.0% |
| | Apr. 27, 2020 | | | | | |
| #44-89 | Apr. 28, 2020- | 46 | 0 | 0.0% | 0 | 0.0% |
| | May 8, 2020 | | | | | |
| #90-218 | May 8, 2020- | 125 | 14 | 11.2% | 5 | 4.0% |
| | Jun. 2, 2020 | | | | | |
| #219-243 | Jun. 5, 2020- | 25 | 3 | 12.0% | 2 | 8.0% |
| | Jun. 12, 2020 | | | | | |
| Total | | 239 | 19 | 7.9% | 7 | 2.9% |

Screening feline serum samples by SARS-CoV-2 RBD-based ELISA. To validate the SARS-CoV-2 N-specific IgG antibody responses in cat sera, we developed an RBD-based IgG ELISA in order to detect RBD-specific antibodies by using recombinant RBD protein. A representative test is shown in FIG. 6B, in which 13 known negative feline sera samples from the N-based ELISA were used to determine the cut-off value shown as a red dashed line. Among the 19 pet cats seropositive of N-specific IgG antibodies (FIG. 6A), 7 samples were found to be weakly to strongly positive for the RBD-specific IgG antibodies in the RBD-based ELISA (FIG. 6B), confirming that these cats have indeed been exposed to SARS-CoV-2.

To compare the performance of N- and RBD-based ELISAs for diagnostic purpose, we simultaneously employed both assays on all 25 cat sera received from Jun. 5 to 12, 2020, along with a few N-seropositive but RBD-seronegative samples as controls. Shown in FIG. 6C, out of 7 sera samples that were N-seropositive, 3 were strongly RBD-seropositive and 1 was weakly RBD-seropositive. More importantly, we did not find any RBD-seropositive but N-seronegative samples, implicating that the N-based ELISA is a more sensitive to detect SARS-CoV-2 exposure in pet cats than the RBD-ELISA, perhaps partly due to the higher levels of N-specific IgG antibodies in the cat sera than the RBD-specific IgG antibodies. As such, we propose that N-based ELISA can be used as an initial (pilot) screening tool for serological evidence of SARS-CoV-2 exposure, which can be followed by the RBD-ELISA as a validation test.

Screening feline serum samples by FIPV-based ELISA. A possible explanation for the presence of N-seropositive but RBD-seronegative cat sera is that the N-specific IgG antibodies present in the cat sera might cross-react with and therefore detect infection of some of the pet cats that have been infected by known feline coronaviruses (FCoVs) that appear to be prevalent among pet cats^{41,42}. These FCoVs include but are not necessarily limited to feline enteric CoV and its pathogenic variant feline infectious peritonitis virus (FIPV). However, SARS-CoV-2 (a betacoronavirus) and FCoVs (alphacoronaviruses) are taxonomically distant and their N proteins share only 26% amino acid identity and 44% amino acid similarity. Regardless, in order to evaluate the potential seroprevalence of FCoVs in our cat sera and to determine the potential cross-reactivity of the coronavirus antibodies, we used commercial antigens from inactivated FIPV particles as outlined in the Materials and Methods section in the FCoV ELISA. Using this FCoV ELISA, we screened 116 cat serum samples, including the 19 N-seropositive sera samples, for potential antibodies that can cross react with FCoVs. As FIPV is the virulent form of the feline enteric CoV, the FCoV ELISA might detect both feline enteric CoV infections as well as FIPV infections. As shown in FIG. 6D, 41 out of 116 feline sera samples (35%) are seropositive for FCoV antibodies, which is consistent with the known widespread FCoV infections in pet cats^{41,42}, and is at a much higher percentage than that of antibodies against the SARS-CoV-2's N protein (7.9%). Taken together, these data suggest that the SARS-CoV-2 N-based ELISA can specifically detect N-specific IgG antibodies that are unlikely to be cross-reactive with the anti-FCoV antibodies in these cats. In support of this notion, as shown in Table 2, among 116 samples tested, the seroprevalence of SARS-CoV-2 N-specific and FCoV-specific IgG antibodies do not seem to correlate well. Four out of nineteen SARS-CoV-2 N-seropositive samples are actually FCoV-seronegative (21.1%), while about 26.8% of SARS-CoV-2 N-seronegative samples are FCoV-seropositive. In addition, the seroprevalence of SARS-CoV-2 N-specific IgG antibodies in the FCoV-seronegative samples (5.3%) is not much different from that of the total samples (7.9%). Taken altogether, our data suggest that the SARS-CoV-2 N-based ELISA can accurately detect N-specific antibodies independently of the potential seroprevalence for FCoV infections in these cats. On the other hand, 78.9% (15 out of 19) of the SARS-CoV-2 N-seropositive samples were seropositive for FCoV, which is a much higher percentage than that of the SARS-CoV-2 N-seronegative samples (26.8%). We surmise that this is partly due to the fact that there are likely many FCoV antigens with a relatively large diversity of epitopes for antibodies in the inactivated feline coronaviruses provided by the commercial source, which might include some of the highly conserved epitopes shared among different coronaviruses. In summary, our results demonstrate a high seroprevalence of FCoV antibodies in these pet cats and higher specificity of N-based ELISA than RBD-based ELISA in detecting SARS-CoV-2 exposure in pet cats.

TABLE 2
Seroprevalence of SARS-CoV-2 N and
FCoV antibodies in feline sera

| | total | N sero- | N sero- | N sero- |
|---|---|---|---|---|
| feline sera | (#) | positive (#) | negative (#) | positive (%) |
| total (#) | 239 | 19 | 220 | 7.9% |
| FCoV sero- | 41 | 15 | 26 | 36.6% |
| positive (#) | | | | |
| FCoV sero- | 75 | 4 | 71 | 5.3% |
| negative (#) | | | | |
| FCoV sero- | 35.3% | 78.9% | 26.8% | |
| positive (%) | | | | |

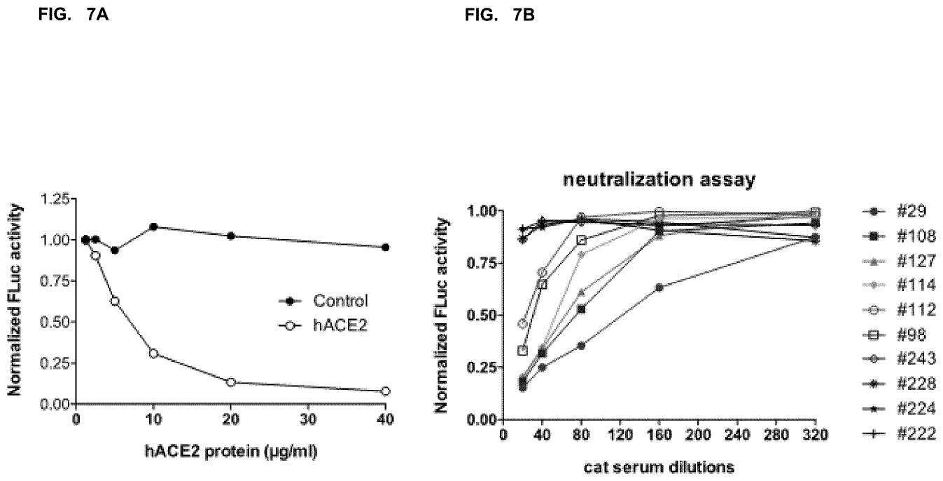

Determination of SARS-CoV-2 neutralization titers in cat sera. To determine whether neutralizing antibodies can be developed in the seropositive pet cats, we conducted a virus neutralization assay to determine whether cat sera could inhibit a recombinant VSV expressing firefly luciferase reporter gene that is pseudotyped with the SARS-CoV-2 spike (S) protein (rVSVΔG/Fluc-CoV2-S)³⁶ from infecting human kidney epithelial 293T cells stably expressing hACE2. To ensure that this assay works effectively, we first incubated cells with a mixture of the reporter virus with increasing concentrations of the recombinant hACE2 protein, which is the host receptor for SARS-CoV2, and showed that there was a dose-dependent inhibition, demonstrating the specificity of this assay to evaluate SARS-CoV-2 entry (FIG. 7A). Using this assay, we next evaluated the neutralizing potential of the selected seropositive cat sera, To do this, serial dilutions of the respective sera samples were incubated with the reporter virus before adding onto the target cells. As shown in FIG. 7B, individual cat serum exhibited differential potential to inhibit the reporter virus entry into cells. Some cat sera (#243, #228, #224, and #222) did not show any neutralizing activity even at the lowest dilution factor (1:20), while other cat serum samples showed various levels of neutralizing activities with the strongest one being detected for serum sample #29. We quantified the neutralization titers for 38 total cat serum samples, including all 19 SARS-CoV-2 N-seropositive samples, 6 N-seronegative but FCoV-seropositive samples, and 13 N- and FCoV-seronegative samples (Table 3). SARS-CoV-2 neutralizing activity was detected in a total of 15 of those cat sera, all of which are N-seropositive and 7 of which are RBD-seropositive. Our results suggest that besides targeting the RBD-associated neutralizing epitopes of the SARS-CoV-2 spike (S) protein, a high percentage of the neutralizing activities in cat sera also targets non-RBD region and that the RBD-based ELISA is less sensitive than the N-based ELISA to detect SARS-CoV-2 associated IgG antibodies in pet cats.

TABLE 3
ELISA results and neutralization titers of feline serum samples

| sample | N | RBD | FCoV | nAb |
|---|---|---|---|---|
| ID | seropositive | seropositive | seropositive | titer |
| 93 | + | + | + | 60 |
| 95 | + | + | + | 40 |
| 98 | + | + | + | 30 |
| 112 | + | + | + | 20 |
| 114 | + | + | + | 50 |
| 233 | + | + | + | 20 |
| 242 | + | + | + | 50 |
| 11 | + | − | − | 60 |
| 29 | + | − | + | 120 |
| 108 | + | − | + | 80 |
| 125 | + | − | − | 80 |
| 127 | + | − | + | 60 |
| 133 | + | − | + | 100 |
| 194 | + | − | + | 120 |
| 231 | + | − | + | 50 |
| 92 | + | − | − | ND |
| 134 | + | − | − | ND |
| 141 | + | − | + | ND |
| 188 | + | − | + | ND |
| 1 | − | − | − | ND |
| 4 | − | − | − | ND |
| 109 | − | − | − | ND |
| 124 | − | − | − | ND |
| 132 | − | − | − | ND |
| 148 | − | − | + | ND |
| 167 | − | − | + | ND |
| 170 | − | − | + | ND |
| 186 | − | − | | ND |
| 192 | − | − | + | ND |
| 193 | − | − | − | ND |
| 201 | − | − | + | ND |
| 206 | − | − | − | ND |
| 222 | − | − | − | ND |
| 224 | − | − | − | ND |
| 228 | − | − | − | ND |
| 234 | − | − | + | ND |
| 241 | − | − | − | ND |
| 243 | − | − | − | ND |

(ND: not detected)

Individual and pooled ELISA screenings of canine serum samples. In addition to feline serum samples, we received canine sera from the same veterinary medical center during the same period of times (Table 4). We first used the SARS-CoV-2 N-based ELISA to screen 212 dog sera received from Apr. 24 to Jun. 2 2020 and identified 4 seropositive samples (1.9%). Due to the relatively low seroprevalence of the N-specific IgG antibodies in dog sera and the need for screening a larger number of pet dog samples than pet cat samples in order to ascertain the results, we decided to develop a pooled ELISA screening method, in which a pool of 5 dog serum samples was used in each test well of the 96-well plate.

TABLE 4
Canine serum samples screened by SARS-CoV-2 N- and RBD-
based ELISAs as well as for SARS-CoV-2 neutralization.

| canine | Collection | N ELISA | N sero- | N sero- | RBD sero- | RBD sero- | nAb |
|---|---|---|---|---|---|---|---|
| serum ID | Date | (#) | positive (#) | positive (%) | positive (#) | positive (%) | titer |
| #1-38 | Apr. 24, 2020- | 37 | 0 | 0.0% | 0 | | |
| | Apr. 27, 2020 | | | | | | |
| #39-108 | Apr. 28, 2020- | 71 | 1 | 1.4% | 0 | | ND |
| | May 3, 2020 | | | | | | |
| #109-410 | May 2, 2020- | 302 | 3 | 1.0% | 0 | | ND |
| | Jun. 2, 2020 | | | | | | |
| #411-511 | Jun. 5, 2020- | 100 | 1 | 1.0% | 0 | | ND |
| | Jun. 12, 2020 | | | | | | |
| Total | | 510 | 5 | 1.0% | 0 | 0.0% | |

(ND: not detected)

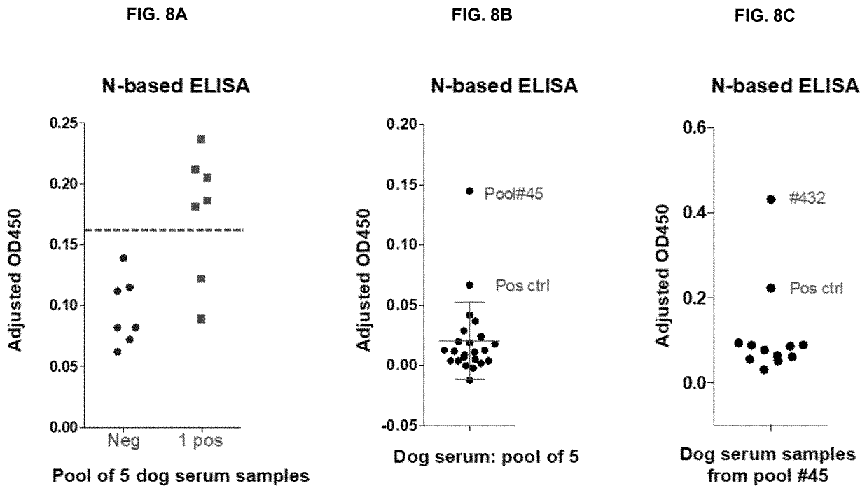

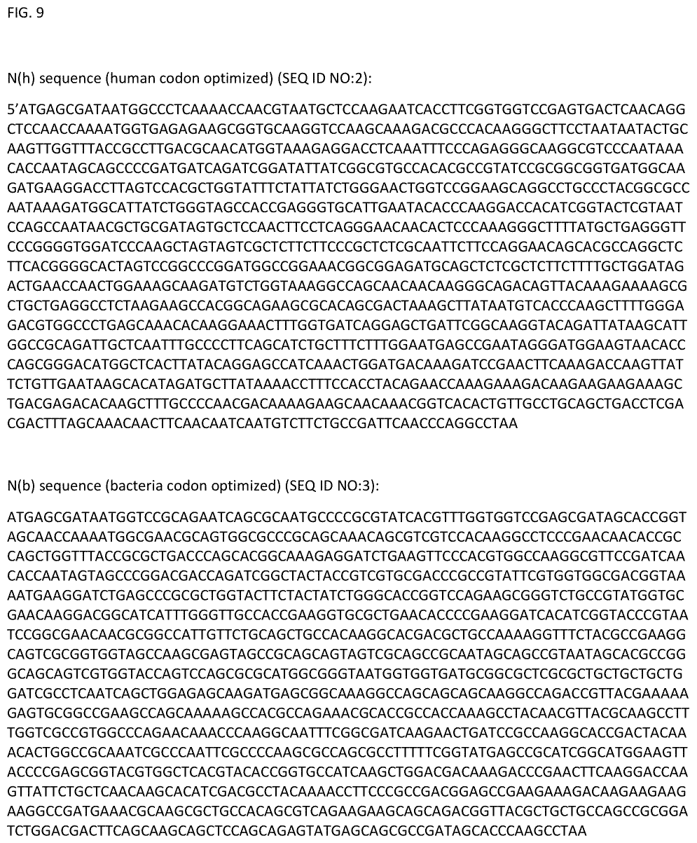

First, in order to determine the sensitivity and specificity of this pooled testing method, we used various known seronegative and seropositive dog's serum samples that have been screened by the N-based ELISA described above to set up seven sets of negative controls with each pool containing five randomly selected N-seronegative pet dog's serum samples, as well as a pool of positive control that consisted of four known N-seronegative pet dog's serum samples and a known N-seropositive dog's serum sample. As shown in FIG. 8A, there was a statistical significance between the positive and negative pooled samples with all seven pooled negative controls showing a consistently low levels of OD₄₅₀ values, while five out of seven (5/7) pooled positive controls showing OD₄₅₀ values that were above the cut-off OD₄₅₀ value of 0.178, suggesting that this pooled sampling method was highly specific and had a sensitivity rate of approximately 71%.

Using this method, we conducted pooled testing of 300 pet dog's serum samples and found that only one pool (#45) was seropositive for N-specific IgG antibodies (FIG. 8B). To confirm the result and to identify the individual dog serum in this pool that is responsible for seropositivity, we conducted individual testing of all five samples in this pool #45 along with several known seronegative dog's samples and one known dog's seropositive sample and identified dog's serum sample #432 as the seropositive sample in this pool of dog's sera (FIG. 8C). From both individual and pooled testing methods, we identified a total of five out of 532 (5/532) N-specific seropositive samples, which implicates a very low seropositivity rate of about 1% (Table 4). It's noteworthy that none of the five N-seropositive pet dog's serum samples were RBD-seropositive in the SARS-CoV-2 RBD-based ELISA or could exhibit any neutralizing activity in the SARS-CoV-2 spike-pseudotyped vesicular stomatitis virus (VSV)-based neutralization assay (Table 4). Collectively, our results demonstrate a relatively low level of seroprevalence of SARS-CoV-2 exposure in pet dogs and a limited neutralizing antibody response in even the few seropositive dog's sera.

Discussion

It has been demonstrated that multiple animal species, including dogs and cats, are susceptible to SARS-CoV-2 infection under experimental conditions or natural settings³⁴. However, little is known about the prevalence of SARS-CoV-2 infection of companion animals in households. We have successfully generated various ELISAs to assess antibodies against different proteins of feline coronaviruses (FCoVs) and SARS-CoV-2, as well as a convenient SARS-CoV-2 spike-pseudotyped vesicular stomatitis virus (VSV)-based neutralization assay to evaluate the levels of neutralizing antibodies in the serum samples of pet cats and pet dogs in the state of Minnesota during the early days of the COVID-19 pandemic (mid-April to mid-June of 2020). Altogether, we analyzed the seroprevalence of SARS-CoV-2 antibodies in 239 pet cats and 510 pet dogs, of which serum samples were collected at a veterinary medical center of the University of Minnesota, Twin Cities during the time when the local human COVID-19 positive cases increased steadily but remained relatively low with 7-day average cases in MN from 2 to 36. Our results showed that the seroprevalence of SARS-CoV-2 in pet cats appeared to be higher (11%-12%) during the period between May 8 and Jun. 12, 2020 than in the earlier days of the pandemic (Apr. 6-Apr. 27, 2020), whereas the seroprevalence in pet dogs during these periods of time remained relatively low (1%).

Our results suggest that pet cats are quite susceptible to natural SARS-CoV-2 infections. These companion animals were presented to the veterinary medical center for various medical conditions or wellness checks. At the time of admission or presentation there was no known or perceived association of COVID-19 disease in these patients and no clinical diagnoses were ultimately consistent with COVID-19 in those that could be reached with a diagnosis, but in some animals, a definitive diagnosis was not reached despite apparent illness. As such, we can neither formally rule in nor rule out COVID-19 disease. Regardless, our study provides the preponderance of evidence for transmission of SARS-CoV-2 from humans to pet cats under natural conditions, however, whether the virus can be transmitted from cats to cats or from cats to humans remains to be determined. As the animal sera used in this study were discarded and archived samples, we could neither determine the status of viral infection in pet cats at the time of admission by viral RNA testing nor were we aware of the owners' health information. A significant percentage of SARS-CoV-2 seropositive cats had digestive signs (vomiting, diarrhea) but rarely respiratory signs at the time of admission. It is therefore less likely that these symptoms are directly caused by SARS-CoV-2 infection, because domestic cats experimentally inoculated with SARS-CoV-2 do not develop any clinical diseases¹⁷⁻¹⁹, suggesting that cats are likely asymptomatic carriers of SARS-CoV-2. That being said, a recent report describes a cat, who lived in the same household with several individuals affected by COVID-19, suffered from severe respiratory distress and thrombocytopenia and was euthanized for humane reasons⁴³. Necropsy results showed that the animal suffered from feline hypertrophic cardiomyopathy and severe pulmonary edema and thrombosis. The cat was found to be PCR-positive for SARS-CoV-2 RNA in its nasal swab, nasal turbinates, and mesenteric lymph node, but there was no evidence of histopathological lesions compatible with a viral infection, suggesting that the animal had a subclinical SARS-CoV-2 infection concomitant with an underlying condition of cardiomyopathy.

Another survey of cats and dogs for SARS-CoV-2 exposures was conducted in households of laboratory-confirmed COVID-19 patients in Besancon, France, during approximately the same period of time as in our study. In that study⁴⁴, the authors collected whole blood samples from 47 pets (34 cats and 13 dogs) that lived in the same households of 31 people known to be PCR-positive for SARS-CoV-2 infections from Jun. 7 to Jun. 12, 2020 (about 2 to 3 months after the owners were clinically diagnosed with COVID-19). Similar to our report, all animals involved in this study appeared to be healthy. Using a combination of 4 tests that included three microsphere immunoassays (MIA) to detect anti-SARS-CoV-2 IgG antibodies produced against the viral N, 51 or S2 spike proteins and a retrovirus (MLV)-based pseudovirus to detect SARS-CoV-2 neutralizing antibodies (similar to our VSV-based pseudovirus neutralization assay), they found a relatively high seroprevalence of SARS-CoV-2 antibodies in 10 of 47 tested animals (21.3%). Specifically, eight out of 34 cats (23.5%) and two out of 13 dogs (15.4%) were found to be seropositive for SARS-CoV-2. They also collected whole blood samples from 16 cats and 22 dogs from households with unknown status of COVID-19 and found that only one cat was tested positive. Their results suggest a much higher level of seropositivity for SARS-CoV-2 among pets from known COVID-19+ households as compared to those of unknown COVID-19 status. The authors concluded that there was an eight times higher risk of testing seropositive for pets in households of COVID-19 positive individuals than for pets co-habilitating in homes with persons of unknown COVID-19 status.

During a similar period of lockdown in Europe (from April to May, 2020), pets from COVID-19 positive owners in Spain were screened for SARS-CoV-2 exposure⁴⁵. Among 23 tested pets that included 8 cats, a guinea pig, 2 rabbits and 12 dogs, only one asymptomatic cat showed positive RT-qPCR results from its oropharyngeal swab, but not from its rectal swab. In a separate European study conducted from March to May, 2020, Patterson and colleagues screened 919 companion animals (540 dogs and 277 cats) living either in northern Italy (mostly in Lombardy) in SARS-CoV-2 positive households or in other geographic areas in Italy that were known to be severely impacted by COVID-19 and did not find any PCR-positive cases⁴⁶. However, 16 of 64 dogs (3.35%) and 6 of 57 cats (3.95%) were positive for anti-SARS-CoV-2 neutralizing antibodies. Among pets living in COVID-19 positive households, 6 of 47 dogs (12.8%) and 1 of 22 cats (4.5%) were found to have anti-SARS-CoV-2 neutralizing antibodies, whereas 1 of 7 dogs (14.3%) and none of 3 cats living in suspected COVID-19 positive households were found to carry neutralizing antibodies. In contrast, much lower levels of anti-SARS-CoV-2 neutralizing antibodies, i.e., 2 of 133 dogs (1.5%) and 1 of 38 cats (2.6%), were found in those pets living in COVID-19 negative households. Therefore, it is clear that pet dogs and cats from COVID-19 positive households are significantly more likely to test positive than those from COVID-19 negative households. Similar to the results of this study, another study showed that none of 10 cats and 9 dogs from owners infected with SARS-CoV-2 in Wuhan, China, during the last two days of the month of March in 2020 were found to be PCR positive for SARS-CoV-2, yet both the ELISA and the plaque reduction neutralization test (PRNT) used to measure titers of SARS-CoV-2 S1-specific IgG and neutralizing antibodies, respectively, in plasma of 2 of the cats and one of the dogs were found to be strongly positive⁴⁷. Taken together, these results reinforce our finding that serology is a sensitive method to screen pets for evidence of SARS-CoV-2 exposures.

Another study conducted in Hong Kong, China from Feb. 11 to Aug. 11, 2020, when 50 cats that lived in the same households with COVID-19 positive owners were screened for evidence of SARS-CoV-2 exposures⁴⁸. Time from onset of COVID-19 symptoms in the pet owners to first sampling of their cats was available for 21 owners of 35 cats and ranged from 3 to 15 (median 8, interquartile range 4) days. SARS-CoV-2 RNA was detected in samples from 6 of 50 cats (12%). In one of the tested households, viral genomes from a cat and its owner were sequenced using the MiSeq sequencing platform after reverse transcription of the viral RNA and shown to be almost completely identical (29,830 nt of the two viral genomes showing 99.8% identity) (accession no. MT628701). This result strongly supports a case for human-to-animal transmission based on the timeline of the virus infections of the cat and its owner and the fact that the cat was strictly homebound.

During the early days of the COVID-19 outbreak in Wuhan, China, serum samples were collected from cats that included 39 pre-epidemic and 102 post-epidemic samples⁴⁹. Fifteen of the 102 sera collected after the outbreak were positive for IgG antibodies against the receptor binding domain (RBD) of SARS-CoV-2 as judged by ELISA. Among those samples, 11 showed evidence for anti-SARS-CoV-2 neutralizing antibodies. Furthermore, no serological cross-reactivity was detected between SARS-CoV-2 and type I or II feline infectious peritonitis virus (FIPV) in that study. Similarly, while we consistently found high prevalence of antibodies against FIPV antigens (35%) in the serum samples of our tested pet cats, a side-by-side comparison of FIPV- and SARS-CoV-2 N-specific IgG antibodies (Tables 2 and 3) suggest that the N-based ELISA did not appear to show any cross-reaction with FCoV antibodies.

In North America, besides our own study, there's only one other study that has been done so far (to the best of our knowledge) to screen companion animals for evidence of SARS-CoV-2 infections. In a longitudinal study of dogs and cats living with at least one SARS-CoV-2 infected human in Texas, the authors of that study found that 47.1% of cats (17) and 15.3% of dogs (59) from 25.6% of the households (39) were positive for SARS-CoV-2 via RT-PCR, viral nucleic acid sequencing or neutralizing antibody assaying⁵⁰. The majority (82.4%) of the infected cats appeared to be asymptomatic. Interestingly, this was a rare example in which the authors were able to successfully isolate virus from one of the naturally infected cats. Only on other study in which SARS-CoV-2 was isolated from one of the two SARS-CoV-2 PCR positive dogs living in close contact with COVID-19 positive patients in Hong Kong, China²³.

It is important to note that our study, the N-based ELISA demonstrates higher levels of specificity and sensitivity to detect SARS-CoV-2 exposures in companion animals than the RBD-based ELISA. Most SARS-CoV-2 serological tests can detect antibodies against the viral nucleocapsid and spike protein (either partial or full-length) and show generally high levels of specificity and sensitivity against SAS-CoV-2 in human sera^{51,52}. However, most if not all of these serological tests have not been carefully evaluated for use in other animal species, including companion animals. Using ELISAs that are based on the full-length RNA-free SARS-CoV-2 N and RBD proteins as well as SARS-CoV-2 nAb assay, we have been able to directly compare the levels of sensitivity and specificity between our N- and RBD-based SARS-CoV-2 ELISAs to detect anti-SARS-CoV-2 IgG antibodies in pet cats and dogs. The RBD-specific IgG ELISA appears to be highly specific, as all 7 RBD seropositive cats are also positive for N-specific IgG and nAbs (Table 3). However, at least 8 RBD-seronegative cat sera (3% of total samples) are positive for both N-specific IgG and nAbs (Table 3), suggesting that the RBD-based ELISA is not very sensitive and fails to detect at least 50% of SARS-CoV-2 exposures in pet cats. On the other hand, our in-house N-based ELISA is highly specific and sensitive to detect SARS-CoV-2 exposure in pet cats and dogs.

As to the level of sensitivity of the N-based ELISA, most of the N-seropositive cat sera (15 out of 19, 79%) have been validated for SARS-CoV-2 exposure as evidenced by the presence of SARS-CoV-2-specific nAbs (Table 3). There are 4 cat sera and 5 dog sera that are N seropositive but RBD seronegative and nAb negative, likely representing mild and limited SARS-CoV-2 antibody responses after low levels of virus exposure in those animals. Correspondingly, human COVID-19 sera have shown variable levels of IgG antibody responses against the viral N and S antigens with distinct kinetics⁵³⁻⁵⁵. Additionally, a significant percentage of the COVID-19 patients exhibit only mild symptoms (20%) and do not develop S1-specific IgG antibodies⁵⁶ or nAbs^{53,54,57}. Taken altogether, our results suggest that SARS-CoV-2 N-based ELISA is a more specific and sensitivity test to detect SARS-CoV-2 exposures in animals such as pet cats and dogs than the RBD-based ELISA.

A comparison of antigen-binding IgG antibodies and nAbs suggests that the level of RBD-binding IgG antibodies is not a reliable assay to evaluate the level of neutralizing capacity in companion animals. Although RBD is the main target of the SARS-CoV-2 neutralizing activity in human sera⁵⁷, other epitopes on the remaining parts of the S protein, such as S1 N-terminal domain (NTD) and S2, can also be targeted by nAbs⁵⁸. We have shown that 50% of cat sera with positive nAbs do not have detectable RBD-specific IgG (Table 3) and that, even though all RBD-seropositive samples exhibit neutralizing capacity, their nAb titers are generally low (1:20 to 1:60), and in some instances, lower than some of the RBD-seronegative samples (1:120). Our results therefore suggest that at least 50% of SARS-CoV-2 neutralizing activity in pet cats targets non-RBD regions of the S protein and caution the exclusive use of RBD-specific IgG antibodies in the evaluation of vaccine-induced immune responses.

In summary, our study provides the first results of serological tests of pet cats and dogs in midwestern USA during the early phase of the COVID-19 pandemic in the state of Minnesota. Our results demonstrate that pet cats are more susceptible to natural SARS-CoV-2 infection than pet dogs in MN early in the COVID-19 pandemic and that SARS-CoV-2 N-based ELISA is more specific and sensitive assay to detect SARS-CoV-2 exposures in pet cats and dogs than the RBD-based ELISA. Further studies will be helpful to monitor the changes of SARS-CoV-2 seroprevalence in companion animals when human COVID-19 positive rate increases and to evaluate the potential and extent of zoonosis and potential reverse zoonosis of SARS-CoV-2 between humans and companion animals.

### CITATIONS FOR EXAMPLE 2

1. Wu F, Zhao S, Yu B, Chen Y-M, Wang W, Song Z-G, Hu Y, Tao Z-W, Tian J-H, Pei Y-Y, et al. A new coronavirus associated with human respiratory disease in China. Nature 2020; 579:265-9.
2. Zhou P, Yang X Lou, Wang X G, Hu B, Zhang L, Zhang W, Si H R, Zhu Y, Li B, Huang C L, et al. A pneumonia outbreak associated with a new coronavirus of probable bat origin. Nature 2020; 579:270-3.
3. Commissioner O of the. Coronavirus (COVID-19) Update: FDA Authorizes Monoclonal Antibodies for Treatment of COVID-19 [Internet]. FDA2020 [cited 2021 Jan. 28]; Available from: https://www.fda.gov/news-events/press-announcements/coronavirus-covid-19-update-fda-authorizes-monoclonal-antibodies-treatment-covid-19
4. Commissioner O of the. COVID-19 Vaccines. FDA [Internet] 2021 [cited 2021 Feb. 27]; Available from: https://www.fda.gov/emergency-preparedness-and-response/coronavirus-disease-2019-covid-19/covid-19-vaccines
5. Vijay R, Perlman S. Middle East respiratory syndrome and severe acute respiratory syndrome. Current Opinion in Virology 2016; 16:70-6.
6. Ye Z W, Yuan S, Yuen K S, Fung S Y, Chan C P, Jin D Y. Zoonotic origins of human coronaviruses. International Journal of Biological Sciences 2020; 16:1686-97.
7. V'kovski P, Kratzel A, Steiner S, Stalder H, Thiel V. Coronavirus biology and replication: implications for SARS-CoV-2. Nature Reviews Microbiology 2020;
8. Walls A C, Park Y-J, Tortorici M A, Wall A, McGuire A T, Veesler D. Structure, Function, and Antigenicity of the SARS-CoV-2 Spike Glycoprotein. Cell 2020; 181:281-292.e6.
9. Hoffmann M, Kleine-Weber H, Schroeder S, Kruger N, Herrler T, Erichsen S, Schiergens T S, Herrler G, Wu N H, Nitsche A, et al. SARS-CoV-2 Cell Entry Depends on ACE2 and TMPRSS2 and Is Blocked by a Clinically Proven Protease Inhibitor. Cell 2020; 181:271-280.e8.
10. Shang J, Ye G, Shi K, Wan Y, Luo C, Aihara H, Geng Q, Auerbach A, Li F. Structural basis of receptor recognition by SARS-CoV-2. Nature 2020; 581:221-4.
11. Wang Q, Zhang Y, Wu L, Niu S, Song C, Zhang Z, Lu G, Qiao C, Hu Y, Yuen K-Y, et al. Structural and Functional Basis of SARS-CoV-2 Entry by Using Human ACE2. Cell 2020; 181:894-904.e9.
12. Wrapp D, Wang N, Corbett K S, Goldsmith J A, Hsieh C-L, Abiona O, Graham B S, McLellan J S. Cryo-EM structure of the 2019-nCoV spike in the prefusion conformation. Science 2020; 367:1260-3.
13. Munir K, Ashraf S, Munir I, Khalid H, Muneer M A, Mukhtar N, Amin S, Ashraf S, Imran M A, Chaudhry U, et al. Zoonotic and reverse zoonotic events of SARS-CoV-2 and their impact on global health. Emerging Microbes and Infections 2020; 9:2222-35.
14. Shi J, Wen Z, Zhong G, Yang H, Wang C, Huang B, Liu R, He X, Shuai L, Sun Z, et al. Susceptibility of ferrets, cats, dogs, and other domesticated animals to SARS-coronavirus 2. Science 2020; 368:1016-20.
15. Chan J F W, Zhang A J, Yuan S, Poon V K M, Chan C C S, Lee A C Y, Chan W M, Fan Z, Tsoi H W, Wen L, et al. Simulation of the Clinical and Pathological Manifestations of Coronavirus Disease 2019 (COVID-19) in a Golden Syrian Hamster Model: Implications for Disease Pathogenesis and Transmissibility. Clinical infectious diseases: an official publication of the Infectious Diseases Society of America 2020; 71:2428-46.
16. Rockx B, Kuiken T, Herfst S, Bestebroer T, Lamers M M, Oude Munnink B B, de Meulder D, van Amerongen G, van den Brand J, Okba N M A, et al. Comparative pathogenesis of COVID-19, MERS, and SARS in a nonhuman primate model. Science 2020; 368:1012-5.
17. Halfmann P J, Hatta M, Chiba S, Maemura T, Fan S, Takeda M, Kinoshita N, Hattori S, Sakai-Tagawa Y, Iwatsuki-Horimoto K, et al. Transmission of SARS-CoV-2 in Domestic Cats. New England Journal of Medicine 2020; 383:592-4.
18. Gaudreault N N, Trujillo J D, Carossino M, Meekins D A, Morozov I, Madden D W, Indran S V., Bold D, Balaraman V, Kwon T, et al. SARS-CoV-2 infection, disease and transmission in domestic cats. Emerging Microbes & Infections 2020; 9:2322-32.
19. Bosco-Lauth A M, Hartwig A E, Porter S M, Gordy P W, Nehring M, Byas A D, VandeWoude S, Ragan I K, Maison R M, Bowen R A. Experimental infection of domestic dogs and cats with SARS-CoV-2: Pathogenesis, transmission, and response to reexposure in cats. Proceedings of the National Academy of Sciences of the United States of America 2020; 117:26382-8.
20. Kim Y Il, Kim S G, Kim S M, Kim E H, Park S J, Yu K M, Chang J H, Kim E J, Lee S, Casel M A B, et al. Infection and Rapid Transmission of SARS-CoV-2 in Ferrets. Cell Host and Microbe 2020; 27:704-709.e2.
21. Imai M, Iwatsuki-Horimoto K, Hatta M, Loeber S, Halfmann P J, Nakajima N, Watanabe T, Ujie M, Takahashi K, Ito M, et al. Syrian hamsters as a small animal model for SARS-CoV-2 infection and countermeasure development. Proceedings of the National Academy of Sciences of the United States of America 2020; 117:16587-95.
22. Schlottau K, Rissmann M, Graaf A, Schön J, Sehl J, Wylezich C, Höper D, Mettenleiter T C, Balkema-Buschmann A, Harder T, et al. SARS-CoV-2 in fruit bats, ferrets, pigs, and chickens: an experimental transmission study. The Lancet Microbe 2020; 1:e218-25.
23. Sit T H C, Brackman C J, Ip S M, Tam K W S, Law P Y T, To E M W, Yu V Y T, Sims L D, Tsang D N C, Chu D K W, et al. Infection of dogs with SARS-CoV-2. Nature 2020; 586:776-8.
24. Newman A, Smith D, Ghai R R, Wallace R M, Torchetti M K, Loiacono C, Murrell L S, Carpenter A, Moroff S, Rooney J A, et al. First Reported Cases of SARS-CoV-2 Infection in Companion Animals—New York, March-April 2020. MMWR Morbidity and Mortality Weekly Report 2020; 69:710-3.
25. Sailleau C, Dumarest M, Vanhomwegen J, Delaplace M, Caro V, Kwasiborski A, Hourdel V, Chevaillier P, Barbarino A, Comtet L, et al. First detection and genome sequencing of SARS-CoV-2 in an infected cat in France. Transboundary and Emerging Diseases 2020; 67:2324-8.
26. USDA APHIS|USDA Confirms SARS-CoV-2 in Mink in Utah [Internet]. [cited 2021 Jan. 27]; Available from: https://www.aphis.usda.gov/aphis/newsroom/stakeholder-info/sa_by_date/sa-2020/sa-08/sare-cov-2-mink
27. Molenaar R J, Vreman S, Hakze-van der Honing R W, Zwart R, de Rond J, Weesendorp E, Smit L A M, Koopmans M, Bouwstra R, Stegeman A, et al. Clinical and Pathological Findings in SARS-CoV-2 Disease Outbreaks in Farmed Mink (Neovison vison). Veterinary Pathology 2020; 57:653-7.
28. Oreshkova N, Molenaar R J, Vreman S, Harders F, Oude Munnink B B, Van Der Honing R W H, Gerhards N, Tolsma P, Bouwstra R, Sikkema R S, et al. SARS-CoV-2 infection in farmed minks, the Netherlands, April and May 2020. Eurosurveillance 2020; 25.
29. WHO|SARS-CoV-2 mink-associated variant strain—Denmark. WHO 2020;
30. Brisse M, Vrba S M, Kirk N, Liang Y, Ly H. Emerging Concepts and Technologies in Vaccine Development. Frontiers in Immunology 2020; 11.
31. Vrba S M, Kirk N M, Brisse M E, Liang Y, Ly H. Development and applications of viral vectored vaccines to combat zoonotic and emerging public health threats. Vaccines 2020; 8.
32. Leroy E M, Ar Gouilh M, Brugère-Picoux J. The risk of SARS-CoV-2 transmission to pets and other wild and domestic animals strongly mandates a one-health strategy to control the COVID-19 pandemic. One Health 2020; 10:100133.
33. McNamara T, Richt J A, Glickman L. A Critical Needs Assessment for Research in Companion Animals and Livestock Following the Pandemic of COVID-19 in Humans. Vector-Borne and Zoonotic Diseases 2020; 20:393-405.
34. Stout A E, André N M, Jaimes J A, Millet J K, Whittaker G R. Coronaviruses in cats and other companion animals: Where does SARS-CoV-2/COVID-19 fit? Veterinary Microbiology 2020; 247:108777.
35. Wan Y, Shang J, Graham R, Baric R S, Li F. Receptor Recognition by the Novel Coronavirus from Wuhan: an Analysis Based on Decade-Long Structural Studies of SARS Coronavirus. Journal of Virology 2020; 94.
36. Schmidt F, Weisblum Y, Muecksch F, Hoffmann H H, Michailidis E, Lorenzi J C C, Mendoza P, Rutkowska M, Bednarski E, Gaebler C, et al. Measuring SARS-CoV-2 neutralizing antibody activity using pseudotyped and chimeric viruses. Journal of Experimental Medicine 2020; 217.
37. Shah V K, Firmal P, Alam A, Ganguly D, Chattopadhyay S. Overview of Immune Response During SARS-CoV-2 Infection: Lessons From the Past. Frontiers in Immunology 2020; 11.
38. Poland G A, Ovsyannikova I G, Kennedy R B. SARS-CoV-2 immunity: review and applications to phase 3 vaccine candidates. The Lancet 2020; 396:1595-606.
39. Lee C Y P, Lin R T P, Renia L, Ng L F P. Serological Approaches for COVID-19: Epidemiologic Perspective on Surveillance and Control. Frontiers in Immunology 2020; 11.
40. COVID-19 Weekly Report—Minnesota Dept. of Health.
41. Haake C, Cook S, Pusterla N, Murphy B. Coronavirus Infections in Companion Animals: Virology, Epidemiology, Clinical and Pathologic Features. Viruses 2020; 12:1023.
42. Zhao S, Li W, Schuurman N, Van Kuppeveld F, Bosch B J, Egberink H. Serological screening for coronavirus infections in cats. Viruses 2019; 11.
43. Segalés J, Puig M, Rodon J, Avila-Nieto C, Carrillo J, Cantero G, Terrón M T, Cruz S, Parera M, Noguera-Julián M, et al. Detection of SARS-CoV-2 in a cat owned by a COVID-19-affected patient in Spain. Proceedings of the National Academy of Sciences of the United States of America 2020; 117:24790-3.
44. Fritz M, Rosolen B, Krafft E, Becquart P, Elguero E, Vratskikh O, Denolly S, Boson B, Vanhomwegen J, Gouilh M A, et al. High prevalence of SARS-CoV-2 antibodies in pets from COVID-19+ households. One Health 2021; 11.
45. Ruiz-Arrondo I, Portillo A, Palomar A M, Santibáñez S, Santibáñez P, Cervera C, Oteo J A. Detection of SARS-CoV-2 in pets living with COVID-19 owners diagnosed during the COVID-19 lockdown in Spain: A case of an asymptomatic cat with SARS-CoV-2 in Europe. Transboundary and Emerging Diseases 2020;
46. Patterson E I, Elia G, Grassi A, Giordano A, Desario C, Medardo M, Smith S L, Anderson E R, Prince T, Patterson G T, et al. Evidence of exposure to SARS-CoV-2 in cats and dogs from households in Italy. Nature Communications 2020; 11:6231.
47. Chen J, Huang C, Zhang Y, Zhang S, Jin M. Severe Acute Respiratory Syndrome Coronavirus 2-Specific Antibodies in Pets in Wuhan, China. Journal of Cleaner Production 2020; 81:e68.
48. Barrs V R, Peiris M, Tam K W S, Law P Y T, Brackman C J, To E M W, Yu V Y T, Chu D K W, Perera R A P M, Sit T H C. SARS-CoV-2 in Quarantined Domestic Cats from COVID-19 Households or Close Contacts, Hong Kong, China. Emerging infectious diseases 2020; 26:3071-4.
49. Zhang Q, Zhang H, Gao J, Huang K, Yang Y, Hui X, He X, Li C, Gong W, Zhang Y, et al. A serological survey of SARS-CoV-2 in cat in Wuhan. Emerging Microbes and Infections 2020; 9:2013-9.
50. Hamer S A, Pauvolid-Correa A, Zecca I B, Davila E, Auckland L D, Roundy C M, Tang W, Torchetti M, Killian M L, Jenkins-Moore M, et al. Title: Natural SARS-CoV-2 infections, including virus isolation, among serially tested cats and dogs in households with confirmed human COVID-19 cases in Texas, USA. bioRxiv 2020; 2020.12.08.416339.
51. Algaissi A, Alfaleh M A, Hala S, Abujamel T S, Alamri S S, Almahboub S A, Alluhaybi K A, Hobani H I, Alsulaiman R M, AlHarbi R H, et al. SARS-CoV-2 S1 and N-based serological assays reveal rapid seroconversion and induction of specific antibody response in COVID-19 patients. Scientific Reports 2020; 10.
52. Ng D L, Goldgof G M, Shy B R, Levine A G, Balcerek J, Bapat S P, Prostko J, Rodgers M, Coller K, Pearce S, et al. SARS-CoV-2 seroprevalence and neutralizing activity in donor and patient blood. Nature Communications 2020; 11.
53. Chen Y, Tong X, Li Y, Gu B, Yan J, Liu Y, Shen H, Huang R, Wu C. A comprehensive, longitudinal analysis of humoral responses specific to four recombinant antigens of SARS-CoV-2 in severe and non-severe COVID-19 patients. PLoS Pathogens 2020; 16.
54. Klein S L, Pekosz A, Park H S, Ursin R L, Shapiro J R, Benner S E, Littlefield K, Kumar S, Naik H M, Betenbaugh M J, et al. Sex, age, and hospitalization drive antibody responses in a COVID-19 convalescent plasma donor population. Journal of Clinical Investigation 2020; 130:6141-50.
55. McAndrews K M, Dowlatshahi D P, Dai J, Becker L M, Hensel J, Snowden L M, Leveille J M, Brunner M R, Holden K W, Hopkins N S, et al. Heterogeneous antibodies against SARS-CoV-2 spike receptor binding domain and nucleocapsid with implications for COVID-19 immunity. JCI Insight 2020; 5.
56. Kowitdamrong E, Puthanakit T, Jantarabenjakul W, Prompetchara E, Suchartlikitwong P, Putcharoen O, Hirankarn N. Antibody responses to SARS-CoV-2 in patients with differing severities of coronavirus disease 2019. PLoS ONE 2020; 15.
57. Wu F, Liu M, Wang A, Lu L, Wang Q, Gu C, Chen J, Wu Y, Xia S, Ling Y, et al. Evaluating the Association of Clinical Characteristics With Neutralizing Antibody Levels in Patients Who Have Recovered From Mild COVID-19 in Shanghai, China. JAMA Internal Medicine 2020; 180:1356.
58. Liu L, Wang P, Nair M S, Yu J, Rapp M, Wang Q, Luo Y, Chan J F W, Sahi V, Figueroa A, et al. Potent neutralizing antibodies against multiple epitopes on SARS-CoV-2 spike. Nature 2020; 584:450-6.

### Example 3

Based on the published SARS-CoV-2 N protein sequence (YP_009724397), two polynucleotide sequences of this gene were chemically synthesized (Twist Biosciences (San Francisco, Calif., USA)) using two separate strategies of codon optimization; one version, called N(h), which was codon optimized for the expression of the recombinant SARS-CoV-2 N protein in human cells and another version of the gene, called N(b) that was codon optimized for the recombinant SARS-CoV-2 N protein expression in bacterial cells. The inventors cloned each version of these synthetic polynucleotide sequences of the SARS-CoV-2 N gene into a bacterial protein expression vector, called pRSF-Duet1, for the sole purpose of expressing recombinant SARS-CoV-2 N proteins with an N-terminal His tag and a C-terminal Strep tag. These two plasmids were called pRSV-CoV2-N(h) and pRSV-CoV2-N(b) and were separately transformed into BL21 bacterial cells using a similar experimental condition as outlined in the Example 1 to express and purify the recombinant N(−RNA) protein that was devoid of associated bacterial RNAs. Unexpectedly, a significantly higher yield of the recombinant N(−RNA) protein from the plasmid encoding the human codon-optimized N(h) gene was obtained compared to the plasmid encoding the bacteria codon-optimized N(b) gene. For instance, they were able to obtain up to 150 mg of the N(−RNA) protein from 1 L of bacteria carrying the pRSV-CoV2-N(h) plasmid, but much less (e.g., greater than 20-fold less) recombinant N(−RNA) protein from the same volume of bacteria that carried the pRSV-CoV2-N(b) plasmid. The two aforementioned codon-optimized polynucleotide sequences (as shown below) share 74.3% sequence identity at the polynucleotide level and encode for the same SARS-CoV-2 N protein. This strongly suggests that the N(h) cDNA sequence may contain some unique polynucleotide sequence combinations that would allow it to produce a much higher yield of the recombinant SARS-CoV-2 N protein in bacteria.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A non-naturally occurring SARS-CoV-2 coronavirus nucleocapsid (N) protein, wherein the N protein is substantially free of RNA.

2.-4. (canceled)

5. A composition comprising the N protein of claim 1, further comprising a pharmaceutically acceptable carrier.

6. (canceled)

7. A method for making the N protein of claim 1 comprising:
exposing N protein to conditions that result in denatured N protein;
isolating the denatured N protein; and
exposing the denatured N protein to conditions that result in refolding of the N protein into a conformation that retains RNA binding activity, wherein the conditions comprise the absence of detectable RNA.

8. The method of claim 7, wherein the denaturing conditions comprise urea.

9. A method comprising:
administering to a subject an amount of the composition of claim 5 effective to induce the subject to produce antibody that specifically binds to the N protein.

10. (canceled)

11. The method of claim 9, wherein the subject is a natural host of SARS-CoV-2, a human, a feline animal, a canine animal, a ferret, a bat, or a pangolin.

12.-13. (canceled)

14. A method for treating an infection in a subject, the method comprising:
administering an effective amount of the composition of claim 5 to a subject having or at risk of having an infection caused by SARS-CoV-2 virus.

15.-18. (canceled)

19. A method of detecting SARS-CoV-2 N protein in a sample comprising:
contacting a biological sample with N protein, wherein the N protein is substantially free of RNA; and
detecting the presence or absence of antibody-antigen complexes formed between the N protein and the antibody present in the biological sample.

20. The method of claim 19, wherein the presence of the complex indicates the subject was exposed to or is currently infected with SARS-CoV-2 virus.

21. The method of claim 19, wherein the absence of the complex indicates the subject was not exposed to or is not infected by SARS-CoV-2 virus.

22. The method of claim 19 wherein the subject displays no signs of SARCoV-2 infection.

23. The method of claim 19, wherein the complex is detected using an anti-IgG antibody.

24. The method of claim 23, wherein the anti-IgG antibody comprises a reporter molecule.

25-26. (canceled)

27. The method of claim 19, wherein the biological sample comprises serum.

28. The method of claim 19, wherein the biological sample is from mammal.

29. The method of claim 28, wherein the mammal is a human, a feline animal, a canine animal, a ferret, a bat, or a pangolin.

30. The method of claim 29, wherein the feline animal is a domesticated cat.

31. The method of claim 29, wherein the canine animal is a domesticated dog.

32. A coding region encoding a N protein comprising SEQ ID NO:1, wherein the coding region is SEQ ID NO:2 or has at least 80% identity to SEQ ID NO:2.

33. (canceled)

34. A genetically modified cell, wherein the cell is an E. coli cell and comprises the coding region of claim 32, and wherein the coding region is optimized for expression in human cells.

35. (canceled)


SEQUENCE LISTING (3 sequences)

SEQ ID NO: 1 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 2 - DNA, 1260 bp, Artificial
  Encodes SEQ ID NO1
atgagcgataatggccctcaaaaccaacgtaatgctccaagaatcaccttcggtggtccgagtgactcaacaggctccaaccaaaatggtgagagaagcggtgcaaggtccaagcaaagacgcccacaagggcttcctaataatactgcaagttggtttaccgccttgacgcaacatggtaaagaggacctcaaatttcccagagggcaaggcgtcccaataaacaccaatagcagccccgatgatcagatcggatattatcggcgtgccacacgccgtatccgcggcggtgatggcaagatgaaggaccttagtccacgctggtatttctattatctgggaactggtccggaagcaggcctgccctacggcgccaataaagatggcattatctgggtagccaccgagggtgcattgaatacacccaaggaccacatcggtactcgtaatccagccaataacgctgcgatagtgctccaacttcctcagggaacaacactcccaaagggcttttatgctgagggttcccggggtggatcccaagctagtagtcgctcttcttcccgctctcgcaattcttccaggaacagcacgccaggctcttcacggggcactagtccggcccggatggccggaaacggcggagatgcagctctcgctcttcttttgctggatagactgaaccaactggaaagcaagatgtctggtaaaggccagcaacaacaagggcagacagttacaaagaaaagcgctgctgaggcctctaagaagccacggcagaagcgcacagcgactaaagcttataatgtcacccaagcttttgggagacgtggccctgagcaaacacaaggaaactttggtgatcaggagctgattcggcaaggtacagattataagcattggccgcagattgctcaatttgccccttcagcatctgctttctttggaatgagccgaatagggatggaagtaacacccagcgggacatggctcacttatacaggagccatcaaactggatgacaaagatccgaacttcaaagaccaagttattctgttgaataagcacatagatgcttataaaacctttccacctacagaaccaaagaaagacaagaagaagaaagctgacgagacacaagctttgccccaacgacaaaagaagcaacaaacggtcacactgttgcctgcagctgacctcgacgactttagcaaacaacttcaacaatcaatgtcttctgccgattcaacccaggcctaa

SEQ ID NO: 3 - DNA, 1260 bp, Artificial
  Encodes SEQ ID NO1
atgagcgataatggtccgcagaatcagcgcaatgccccgcgtatcacgtttggtggtccgagcgatagcaccggtagcaaccaaaatggcgaacgcagtggcgcccgcagcaaacagcgtcgtccacaaggcctcccgaacaacaccgccagctggtttaccgcgctgacccagcacggcaaagaggatctgaagttcccacgtggccaaggcgttccgatcaacaccaatagtagcccggacgaccagatcggctactaccgtcgtgcgacccgccgtattcgtggtggcgacggtaaaatgaaggatctgagcccgcgctggtacttctactatctgggcaccggtccagaagcgggtctgccgtatggtgcgaacaaggacggcatcatttgggttgccaccgaaggtgcgctgaacaccccgaaggatcacatcggtacccgtaatccggcgaacaacgcggccattgttctgcagctgccacaaggcacgacgctgccaaaaggtttctacgccgaaggcagtcgcggtggtagccaagcgagtagccgcagcagtagtcgcagccgcaatagcagccgtaatagcacgccgggcagcagtcgtggtaccagtccagcgcgcatggcgggtaatggtggtgatgcggcgctcgcgctgctgctgctggatcgcctcaatcagctggagagcaagatgagcggcaaaggccagcagcagcaaggccagaccgttacgaaaaagagtgcggccgaagccagcaaaaagccacgccagaaacgcaccgccaccaaagcctacaacgttacgcaagcctttggtcgccgtggcccagaacaaacccaaggcaatttcggcgatcaagaactgatccgccaaggcaccgactacaaacactggccgcaaatcgcccaattcgccccaagcgccagcgcctttttcggtatgagccgcatcggcatggaagttaccccgagcggtacgtggctcacgtacaccggtgccatcaagctggacgacaaagacccgaacttcaaggaccaagttattctgctcaacaagcacatcgacgcctacaaaaccttcccgccgacggagccgaagaaagacaagaagaagaaggccgatgaaacgcaagcgctgccacagcgtcagaagaagcagcagacggttacgctgctgccagccgcggatctggacgacttcagcaagcagctccagcagagtatgagcagcgccgatagcacccaagcctaa